(19) United States
(12) Patent Application Publication
(10) Pub. No.: US 2022/0125866 A1
(43) Pub. Date: Apr. 28, 2022

(54) PHARMACEUTICAL COMPOSITIONS TO ENHANCE PHAGOCYTOSIS WITHOUT INFLAMMATION

(71) Applicant: NANTCELL, INC, Culver City, CA (US)

(72) Inventors: Philip T. LIU, Culver City, CA (US); Kayvan NIAZI, Culver City, CA (US)

(21) Appl. No.: 17/613,602

(22) PCT Filed: May 20, 2020
(86) PCT No.: PCT/US2020/033808

§ 371 Date: Nov. 23, 2021

Related U.S. Application Data
(60) Provisional application No. 62/868,690, filed on Jun. 28, 2019.
(60) Provisional application No. 62/874,376, filed on Jul. 15, 2019.
(60) Provisional application No. 62/874,386, filed on Jul. 15, 2019.

(51) Int. Cl. A61K 36/064; A61K 38/10; A61K 38/08; C12N 15/86

(52) U.S. Cl. CPC A61K 36/064; A61K 38/10; C12N 2710/10341; C12N 15/86; A61K 38/08

(57) ABSTRACT

The invention relates to a composition comprising a peptide and an immunotherapeutic composition, and a method of inducing phagocytosis without inflammation comprising administering the composition.

### CROSS-REFERENCE TO RELATED APPLICATIONS

This application claims the benefit of priority under 35 U.S.C. § 119(e) to U.S. Provisional Application Ser. Nos. 62/868,690, filed Jun. 28, 2019, 62/874,376, filed Jul. 15, 2019 and 62/874,386, filed Jul. 15, 2019. The entire disclosures of each of U.S. Provisional Application Serial Nos. 62/868,690, filed Jun. 28, 2019, 62/874,376, filed Jul. 15, 2019 and 62/874,386, filed Jul. 15, 2019 are incorporated herein by reference.

### BACKGROUND

Peptides are short linear chains of amino acids. They are usually <50 amino acids in length and are often stabilized by disulfide bonds (Hayashi M A et al. Natural Peptides with Potential Applications in Drug Development, Diagnosis, and/or Biotechnology. Int J Pept. 2012;2012:757838). They are designed by rational methods with high specificity to bind and modulate a protein interaction of interest. Many sequences, structures and pattern interactions of oncogenic proteins are available; and as such peptides can be designed specifically as an inhibitor of these interactions (Bidwell G L and Raucher D. Therapeutic peptides for cancer therapy. Part I—peptide inhibitors of signal transduction cascades. Expert Opin Drug Deliv. 2009;6(10):1033-47) for example, if an interaction of two proteins is known, a peptide can inhibit this interaction provided if the sequence of the binding site is known (Draeger L J and Mullen G P. Interaction of the bHLH-zip domain of c-Myc with H1-type peptides. Characterization of helicity in the H1 peptides by NMR. J Biol Chem. 1994;269(3):1785-93). If a protein-protein interaction site is unknown, a series of overlapping peptides of the desired protein are synthesized and can be tested for their capability to bind and inhibit this target interaction (Chen I T, et al. Characterization of p21Cip1/WafI peptide domains required for cyclin E/Cdk2 and PCNA interaction. Oncogene. 1996;12(3′):595-607). The peptide sequence can also be modulated easily, due to their ease of synthesis either by chemical or molecular biological techniques (Bidwell GL ., et al. 2009).

RP-182 is composed of alternating hydrophobic and hydrophilic amino acids, and is not specific to CD206 (also known as mannose receptor), as it also binds Re1B (a transcription factor), signal-regulatory protein alpha (SIRP-a), cluster of differentiation 47 (CD47) and transglutaminase 2 (TGM2) (Scodeller, P., et al. Precision Targeting of Tumor Macrophages with a CD206 Binding Peptide Scientific Reports volume 7, Article number:14655(2017)). In addition, RP-182 is referred to as an anti-inflammatory peptide (U.S. Publication No. 2016/0101150).

Therapeutic peptides, such as RP-182, have several important advantages over proteins or antibodies: they are small in size, easy to synthesize and have the ability to penetrate the cell membranes. They also have high activity, specificity and affinity; minimal drug-drug interaction; and biological and chemical diversity. An added benefit of using peptides as a treatment is that they do not accumulate in specific organs (e.g. kidney or liver), which can help to minimize their toxic side effects (Ali R, et al. New Peptide Based Therapeutic Approaches. In: Ghulam Md A, Ishfaq Ahmed S, editors. Advances in Protein Chemistry. Jeddah: OMICS Group eBooks; 2013). They can also be rapidly synthesized and easily modified (Boohaker R J, et al. The use of therapeutic peptides to target and to kill cancer cells. Curr Med Chem. 2012;19(22):3794-804) and are less immunogenic than recombinant antibodies or proteins (McGregor D P. Discovering and improving novel peptide therapeutics. Curr Opin Pharmacol. 2008;8(5):616-9). Therapeutic peptides show great potential in the treatment of many diseases. In the case of cancer, these peptides can be used in a variety of ways, including carrying cytotoxic drugs, vaccines, hormones and radionuclides (Thundimadathil J. Cancer Treatment Using Peptides: Current Therapies and Future Prospects. J Amino Acids. 2012;2012:Article ID 967347).

RP-182 is one of a series of short peptides that mimic certain bacterial surface molecules. Phagocytosis is a process by which antigen presenting cells take up antigens from the environment as a preliminary to instigating an immune response against the antigen. While many substances are known in the prior art to enhance phagocytosis (and thus enhance antigen presentation), these prior art substances all simultaneously enhance inflammation. Surprisingly and advantageously as disclosed herein, it has been found that RP-182 and related peptides enhance phagocytosis by antigen presenting cells without also upregulating inflammation. This makes RP182 and related peptides useful in various vaccines.

### SUMMARY

One embodiment of the invention relates to a pharmaceutical composition comprising a peptide and an immunotherapeutic composition comprising a viral vector and a nucleic acid sequence encoding an antigen; wherein the peptide is 3 to 24 amino acid residues in length and comprises a striapathic region consisting of alternating hydrophilic and hydrophobic modules; wherein each hydrophilic module consists of from 1 to 5 hydrophilic amino acid residues; and wherein each hydrophobic module consists of from 1 to 5 hydrophobic amino acid residues.

Another embodiment of the invention relates to a method of inducing phagocytosis without inflammation, comprising administering to a subject in need thereof, an immunotherapeutic composition comprising a viral vector and a nucleic acid sequence encoding an antigen; and peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues.

In one aspect, the viral vector is a replication defective adenovirus vector comprising a deletion in an E2b region of the replication defective adenovirus vector and a nucleic acid sequence encoding an antigen.

In another aspect, the replication defective adenovirus vector further comprises a deletion in an E1 region of the replication defective adenovirus vector, a deletion in an E3 region of the replication defective adenovirus vector, a deletion in an E4 region of the replication defective adenovirus vector, or a combination thereof.

In still another aspect, the antigen is selected from the group consisting of a cancer associated antigen, and an infectious disease associated antigen. Another embodiment of the invention relates to a method of inducing phagocytosis, comprising administering to a subject in need thereof, a composition comprising a yeast lysate prepared from a yeast; and an immunotherapeutic composition comprising viral a vector and a nucleic acid sequence encoding an antigen. In one aspect of this embodiment, the method of inducing phagocytosis does not cause inflammation. In one aspect of this embodiment, the yeast lysate lacks yeast membranes and yeast cell walls. In still another aspect of this embodiment, the yeast lysate comprises intact yeast. In a further aspect this embodiment, the yeast is heat-inactivated. In yet another aspect this embodiment, the yeast is selected from the group consisting of Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica. In a preferred embodiment, the yeast is Saccharomyces cerevisiae. In yet another aspect this embodiment, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In another aspect this embodiment, the antigen is selected from the group consisting of a cancer associated antigen and an infectious disease associated antigen.

Another embodiment of the invention relates to a method of inducing phagocytosis, comprising administering to a subject in need thereof, a composition comprising a bacterial lysate prepared from a bacteria; and an immunotherapeutic comprising a viral vector and a nucleic acid sequence encoding an antigen. In one aspect this embodiment, the method of inducing phagocytosis does not cause inflammation. In one aspect this embodiment, the bacteria is selected from the group consisting of Haemophilus influenzae, Klebsiella pneumoniae, Moraxella catarrhalis, Staphylococcus aureus, Streptococcus mitis, Streptococcus pneumoniae, S. pyogenes, K. pneumoniae, M catarrhalis S. pyogenes, S. agalactiae, K ozaenae, S. viridans, Escherichia coli, S. epidermidis, S. salivarius, Corynebacterium pseudodiphtheriticum. In still another aspect this embodiment, the bacteria is a gram negative bacteria (such as cyanobacteria, spirochaetes and proteobacteria). In yet another aspect this embodiment, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In another aspect this embodiment, the antigen is selected from the group consisting of a cancer associated antigen and an infectious disease associated antigen. Another embodiment of the invention relates to a pharmaceutical composition comprising a yeast lysate prepared from a yeast; and a viral vector and a nucleic acid sequence encoding an antigen. In one aspect of this composition, the yeast lysate lacks yeast membranes and yeast cell walls. In still another aspect of the composition, the yeast lysate comprises intact yeast. In a further aspect, the yeast is heat-inactivated. In yet another aspect of the composition, the yeast is selected from the group consisting of Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia hpolytica. In a preferred embodiment, the yeast is Saccharomyces cerevisiae. In still another aspect, the composition further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In one aspect of the composition, the antigen is selected from the group consisting of a cancer associated antigen and an infectious disease associated antigen.

Another embodiment of the invention relates to a pharmaceutical composition comprising a bacterial lysate prepared from a bacteria; and a viral vector and a nucleic acid sequence encoding an antigen. In one aspect of the composition, the bacteria is selected from the group consisting of Haemophilus influenzae, Klebsiella pneumoniae, Moraxella catarrhalis, Staphylococcus aureus, Streptococcus mitis, Streptococcus pneumoniae, S. pyogenes, K. pneumoniae, M catarrhalis S. pyogenes, S. agalactiae, K. ozaenae, S. viridans, Escherichia coli, S. epidermidis, S. salivarius, Corynebacterium pseudodiphtheriticum. In another aspect, the bacteria is a gram-negative bacteria (such as cyanobacteria, spirochaetes and proteobacteria). In still another aspect, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In one aspect of the composition, the antigen is selected from the group consisting of a cancer associated antigen and an infectious disease associated antigen.

### BRIEF DESCRIPTION OF THE DRAWINGS

FIGS. 1A and 1B show NLP04 monocyte-derived dendritic cells (moDC) (GM/IL4) express CD206.

FIGS. 2A-2H show NLP04 moDC stimulated with RP-182 increases adenovirus (AdV)-green fluorescent protein (GFP) efficiency.

FIGS. 3A-3H show NLP04 moDC stimulated with RP-182 increases AdV-GFP efficiency.

FIG. 4 shows NLP04 moDC stimulated with RP-182 induce low levels of IL-8 and 11-6.

FIGS. 5A and 5B show NLP04 moDC stimulated with RP-182 and derivatives then infected with AdV-tbs-GFP.

FIG. 6A and 6B show NLP04 moDC stimulated with RP-182 and derivatives then infected with AdV-tbs-GFP.

FIGS. 7A-7G show NLP04 moDC stimulated with RP-182 and other adjuvants then infected with AdV-tbs-GFP. First data set in each graph (no virus); second data set in each graph (AdV-tbs-GFP).

FIG. 8 shows NLP04 moDC stimulated with RP-182 and other adjuvants then infected with AdV-tbs-GFP.

FIGS. 9A-9E show NLP04 moDC stimulated with RP-182, CC and BCG then infected with AdV-tbs-GFP. “CC” is an E. coli strain and “BCG”=Bacillus Calmette-Guérin. First data set in each graph (no virus); second data set in each graph (AdV-GFP).

FIGS. 10A-10E show NLP04 moDC stimulated with RP-182, CC and BCG then infected with AdV-tbs-GFP.

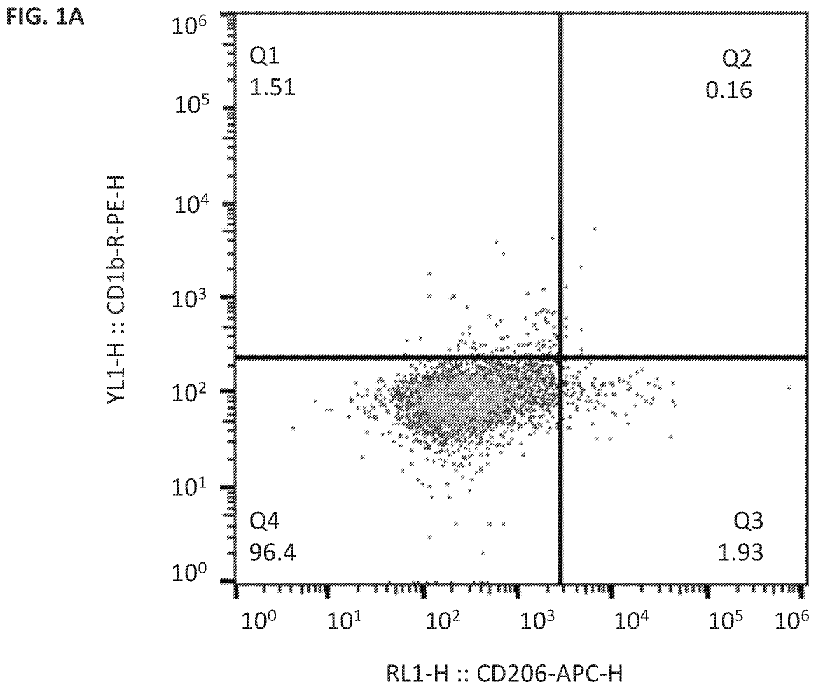

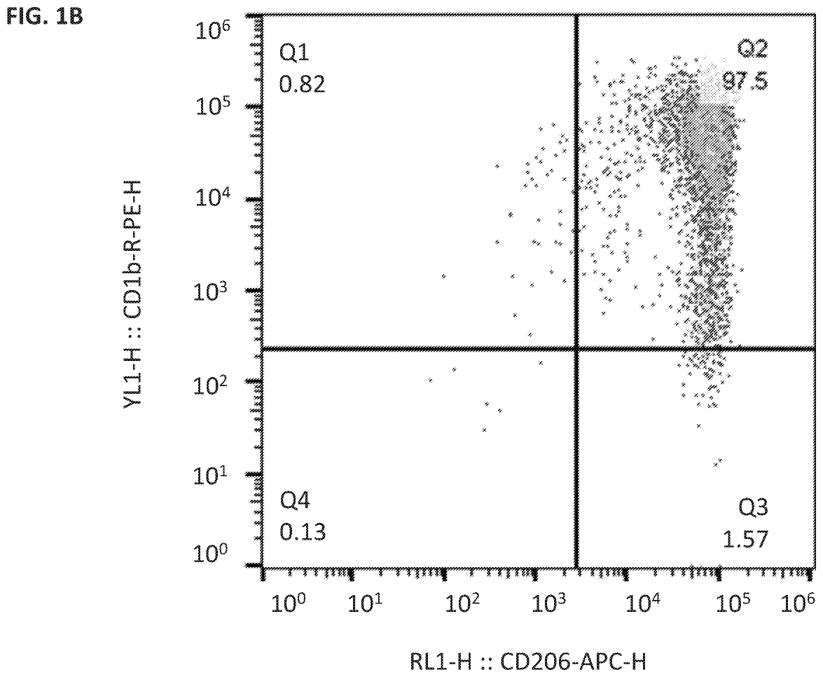

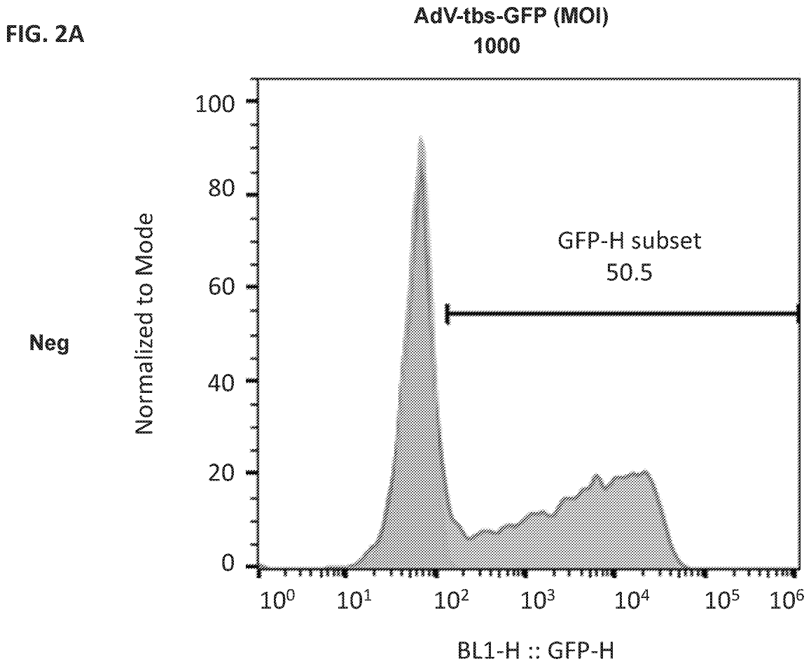

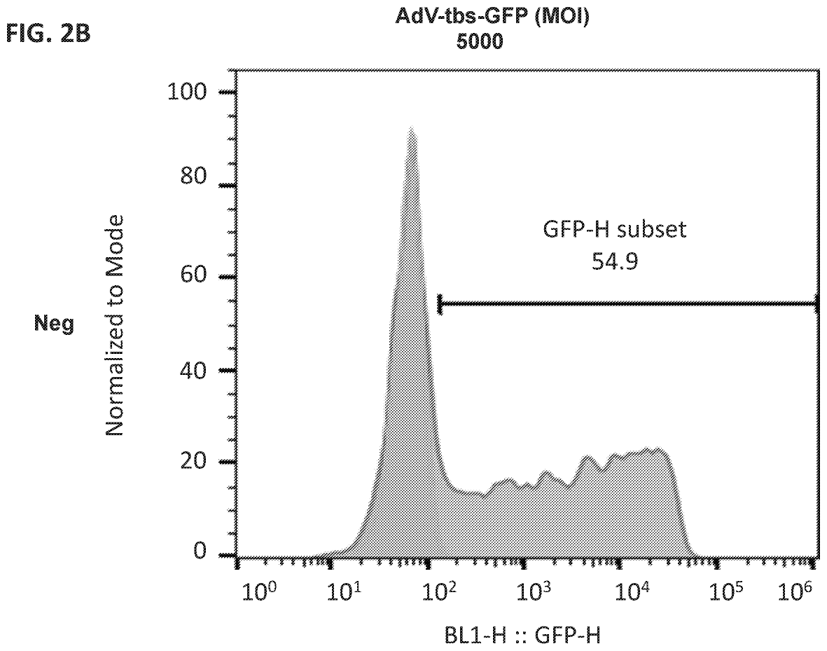

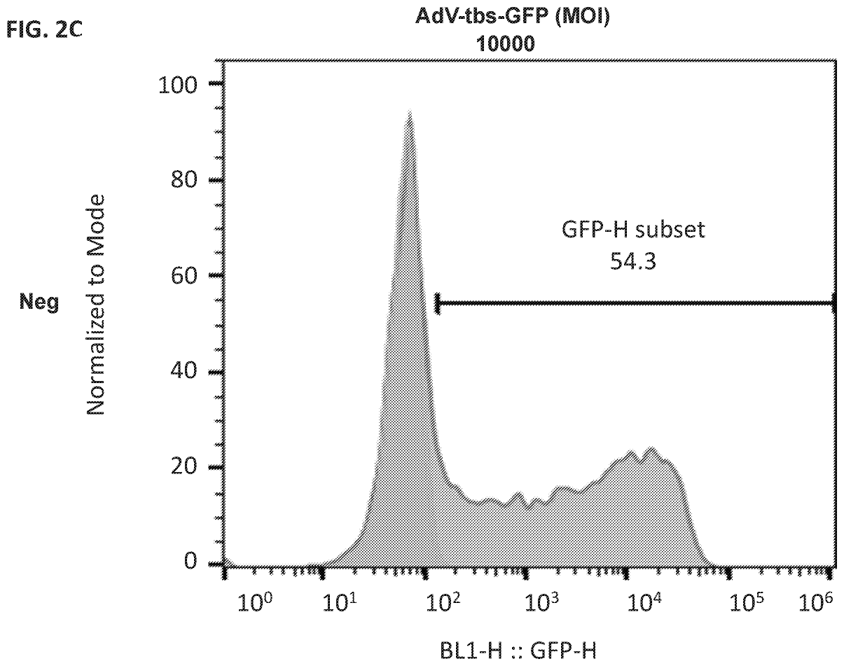

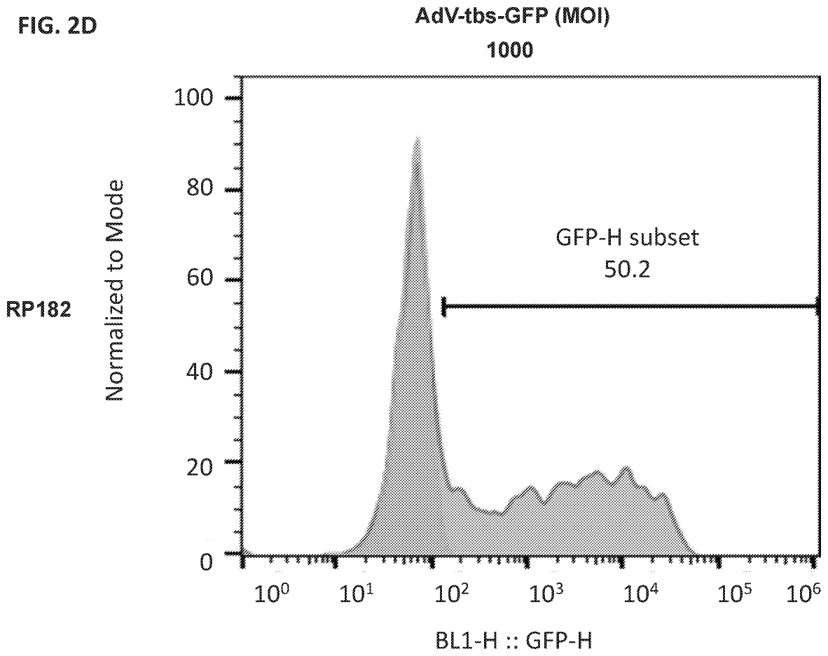

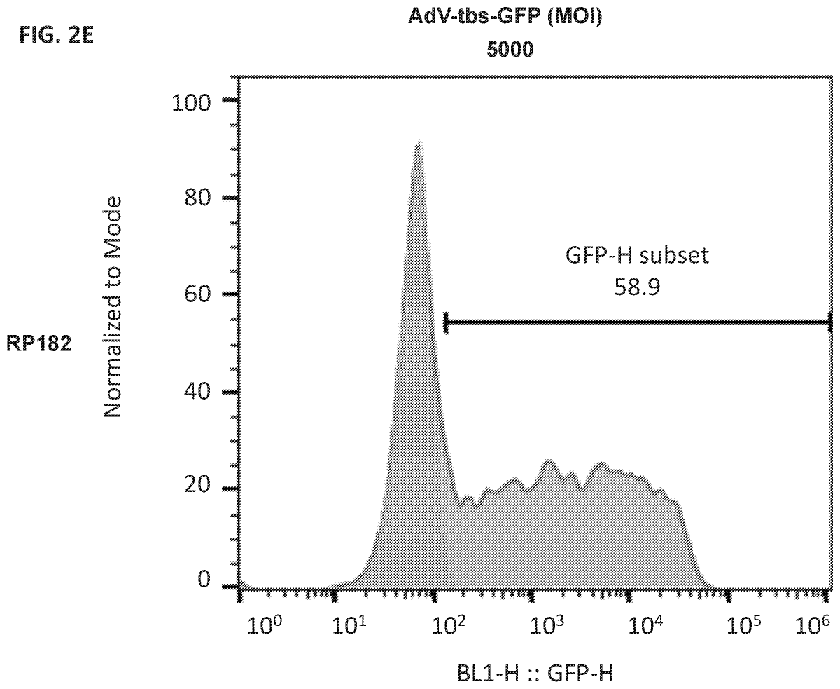

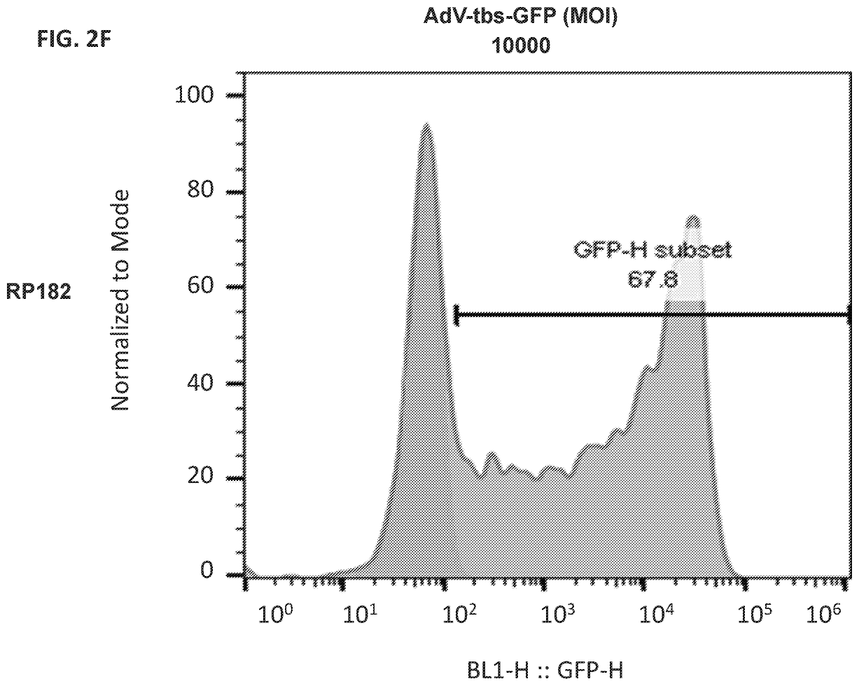

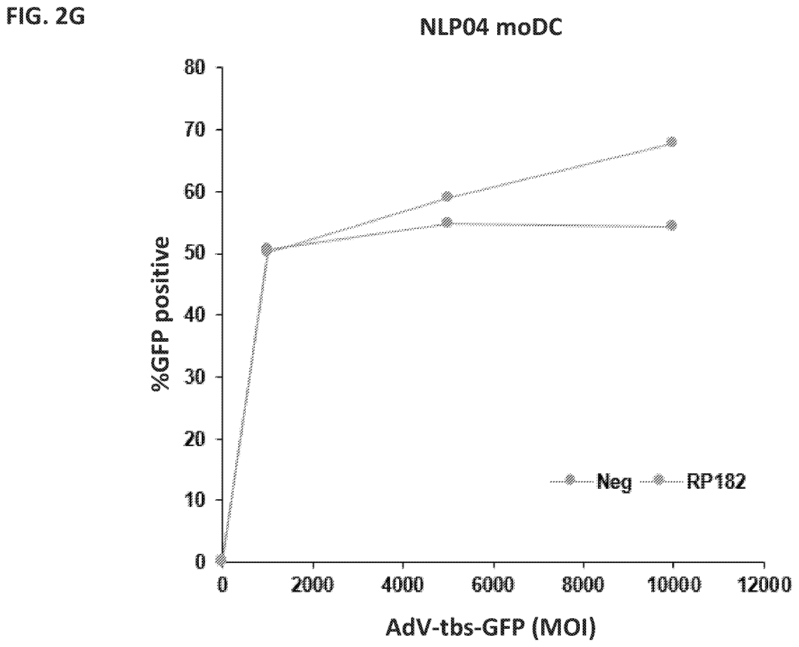

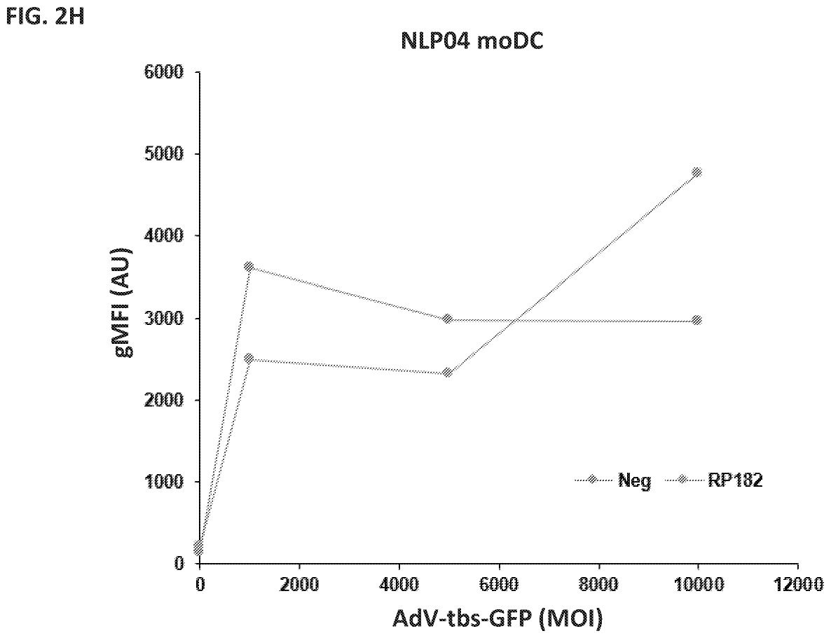

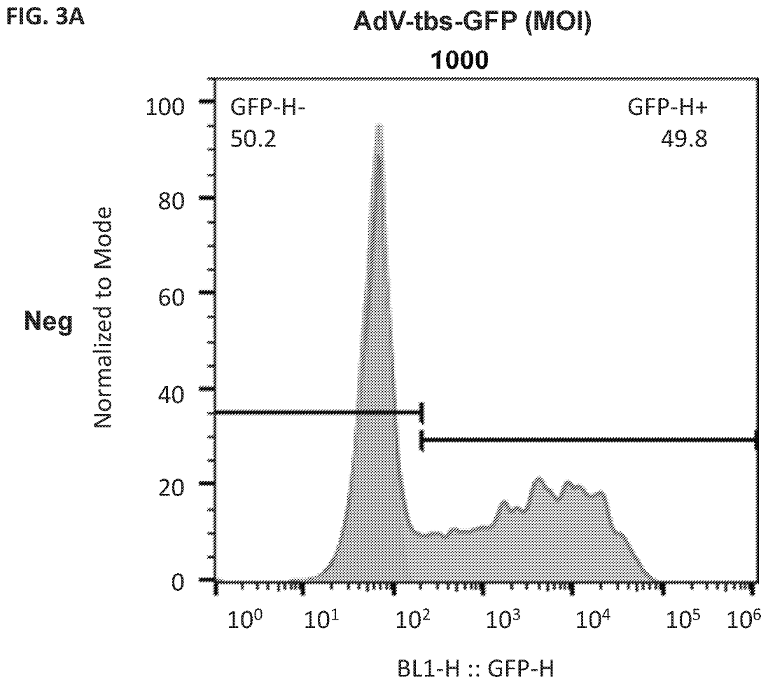

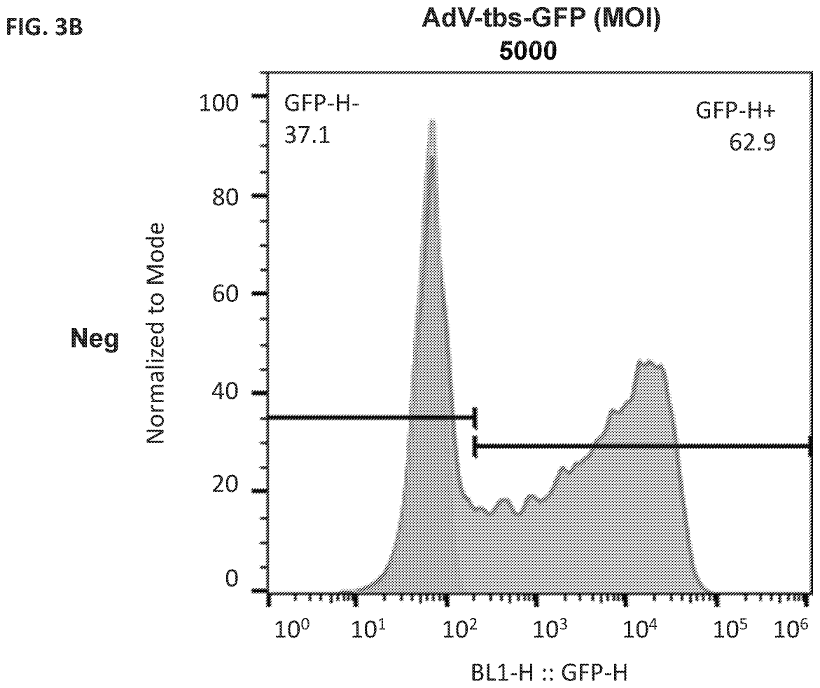

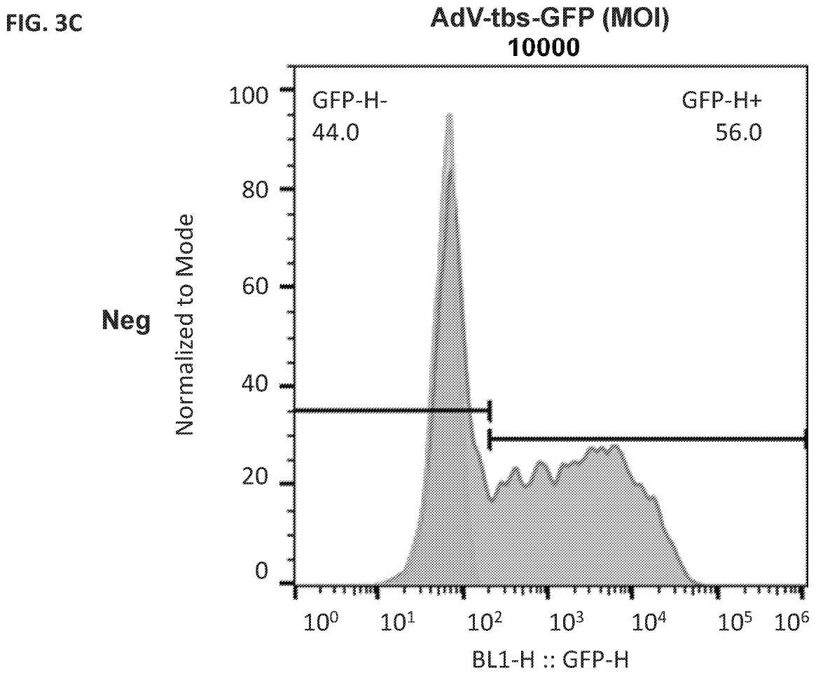

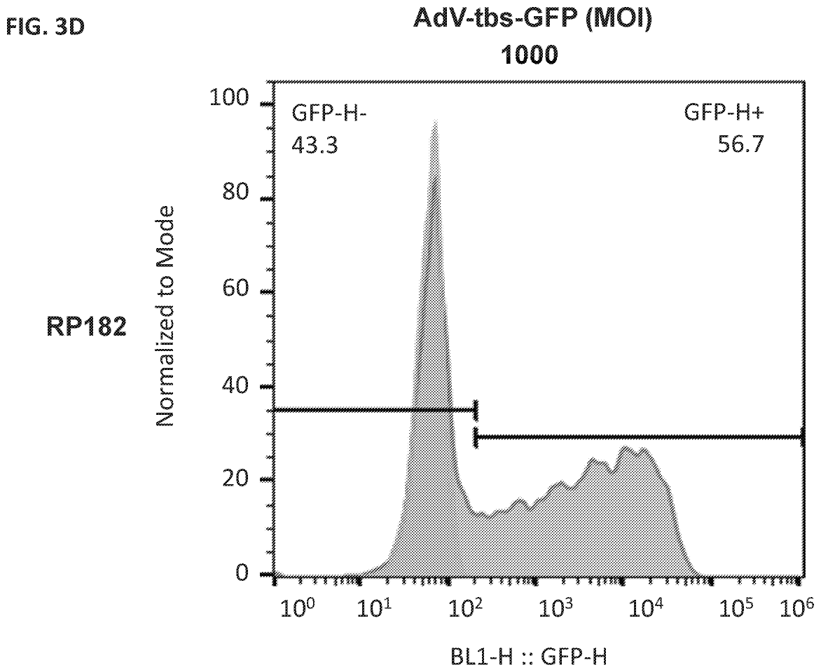

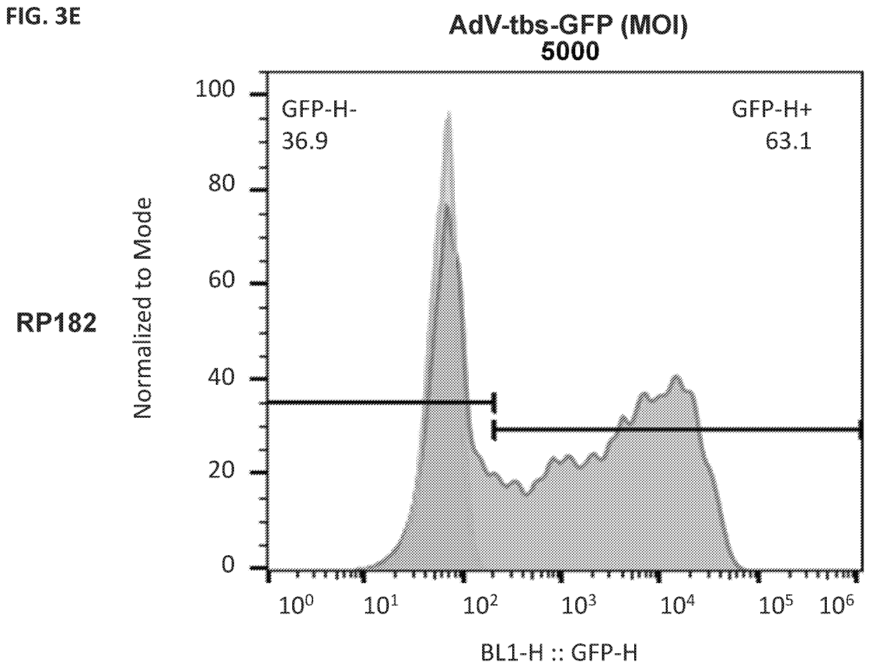

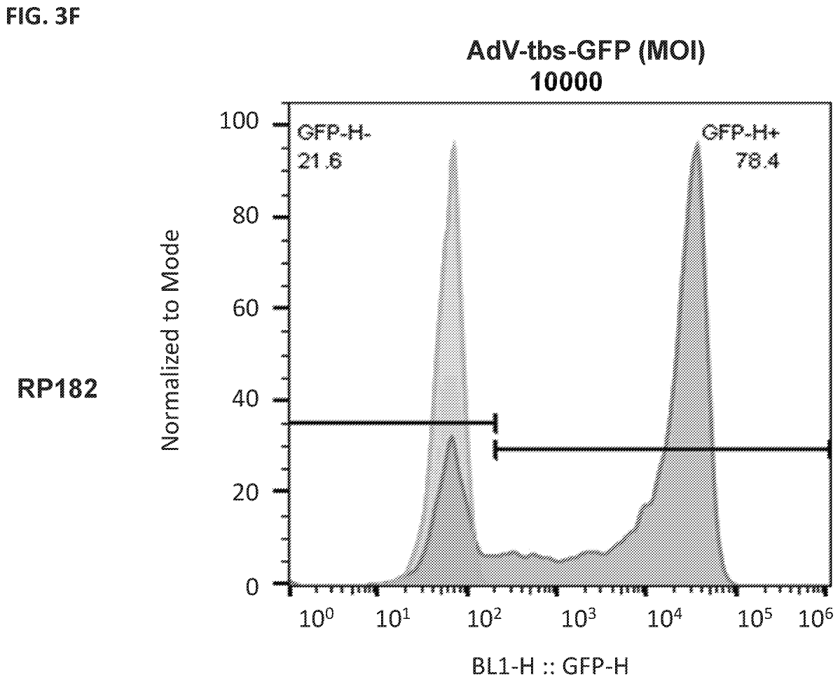

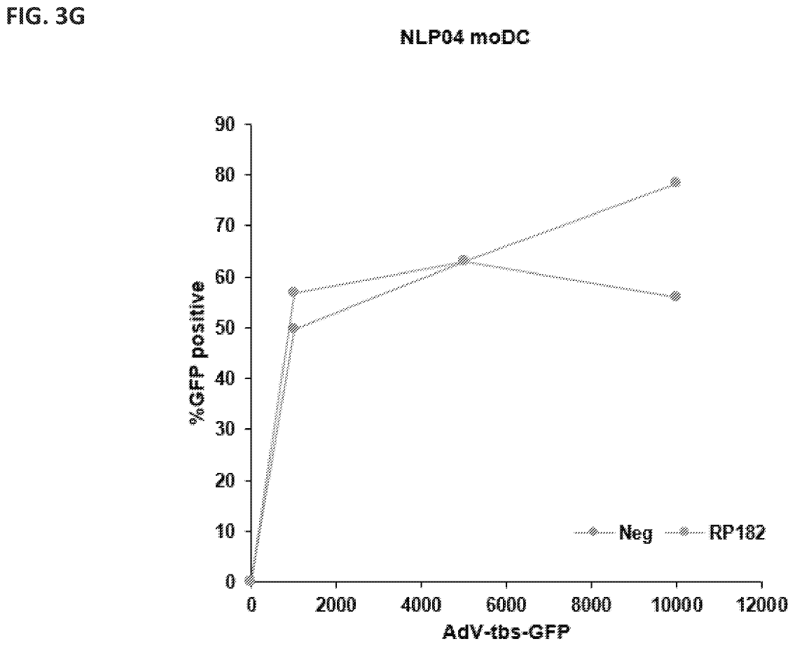

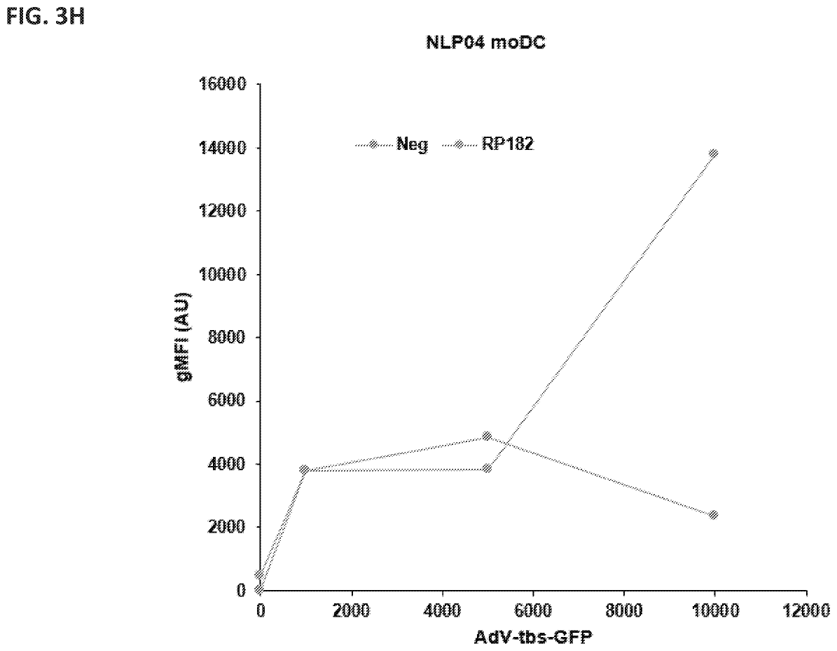

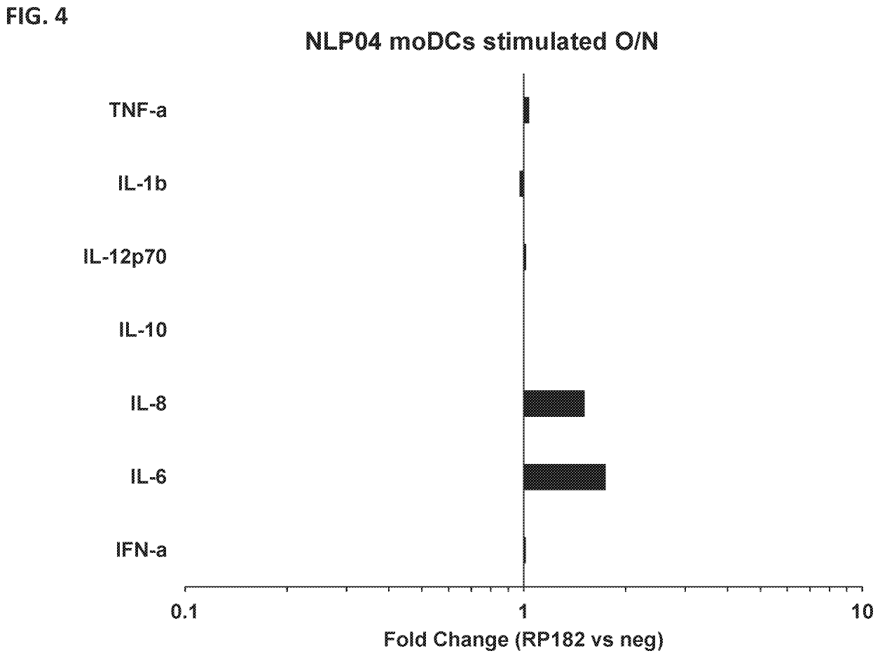

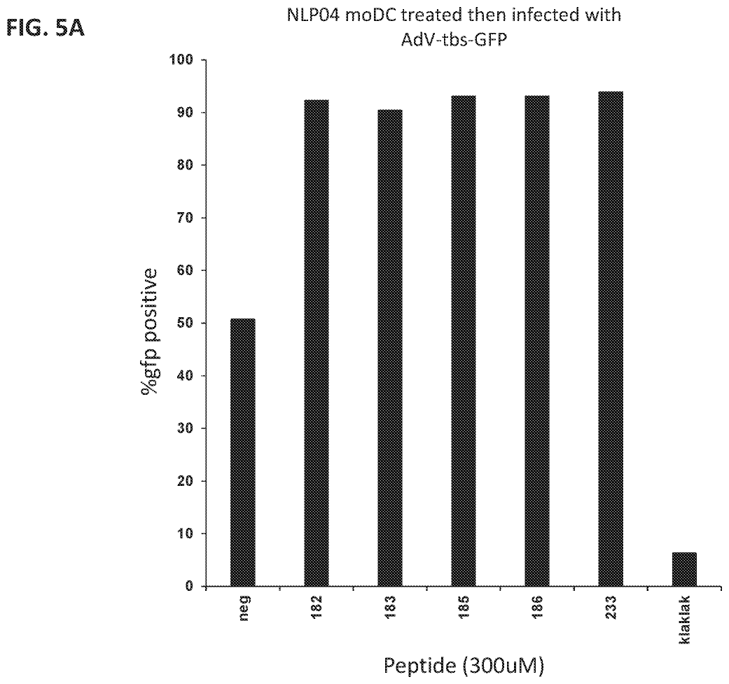

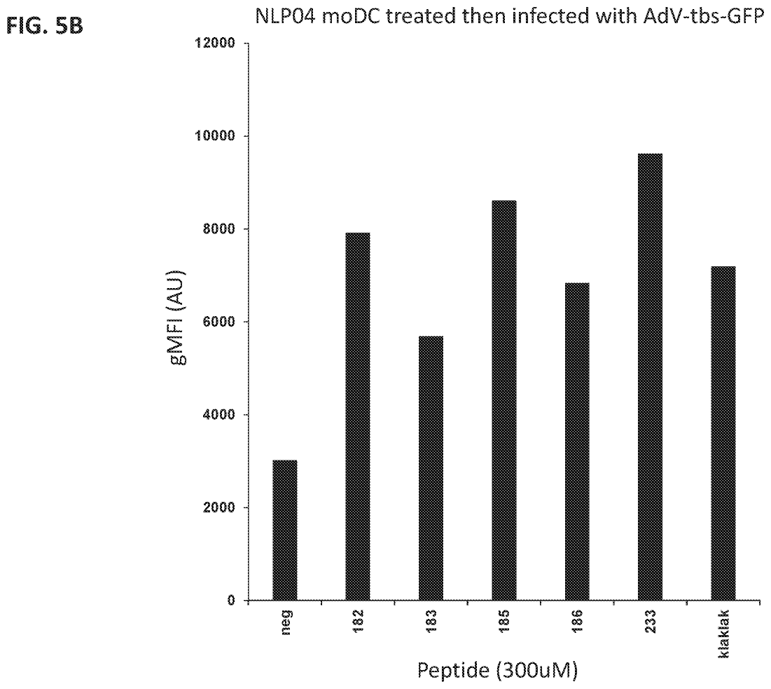

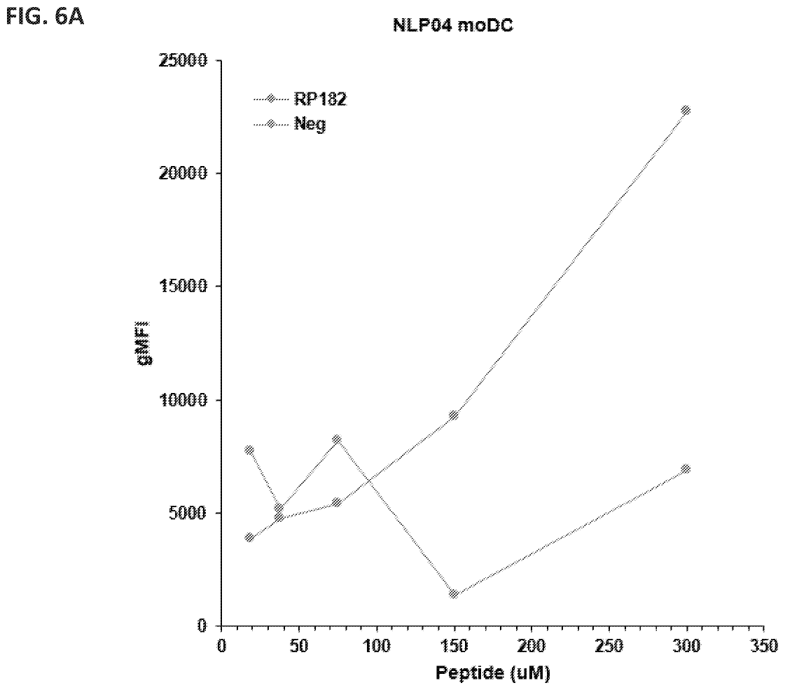

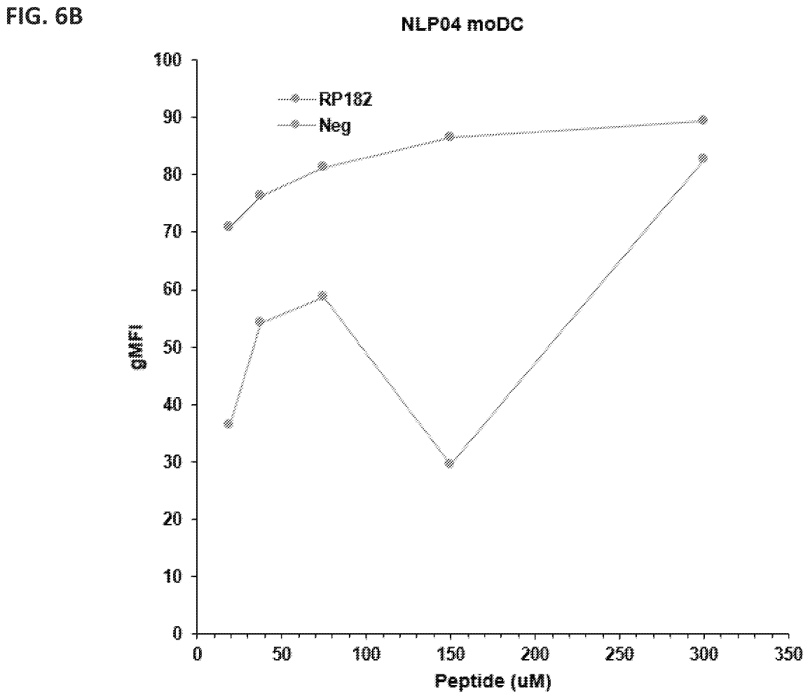

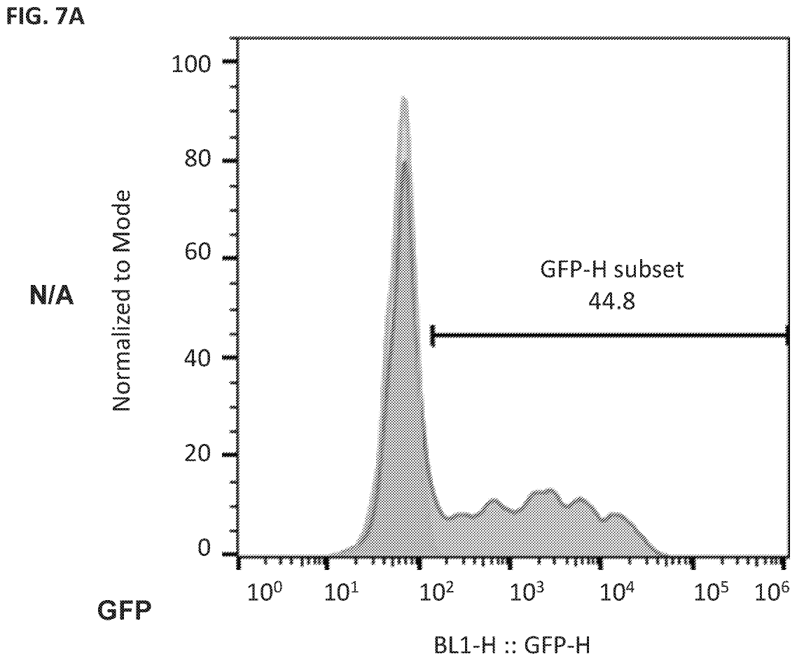

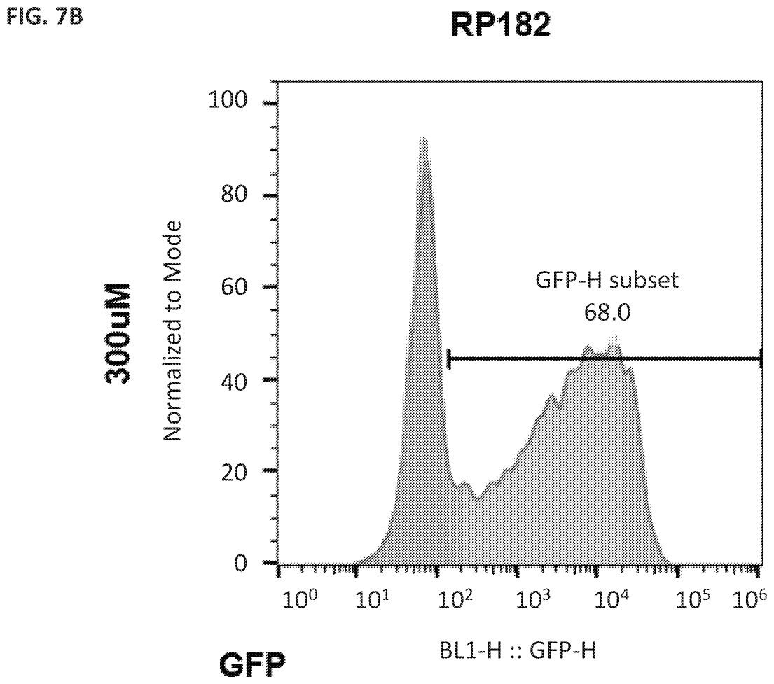

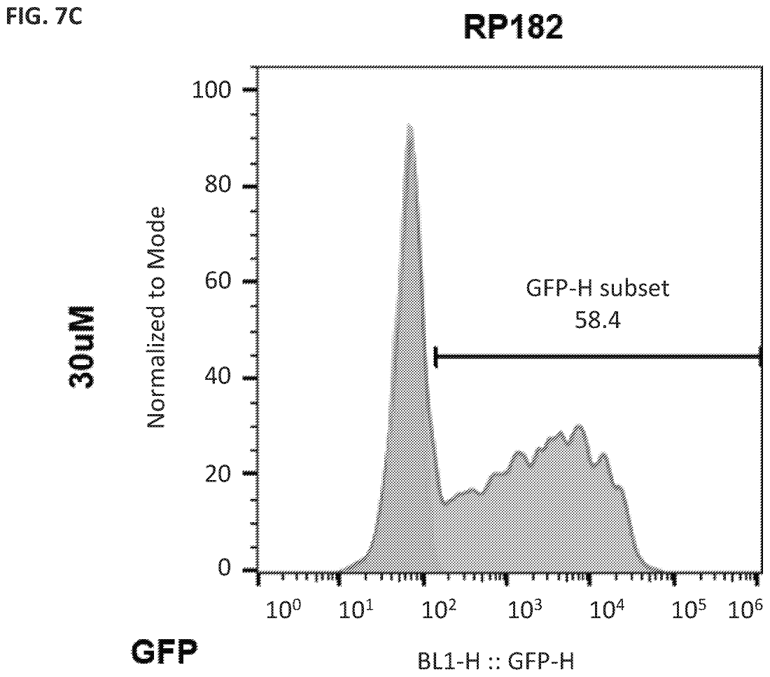

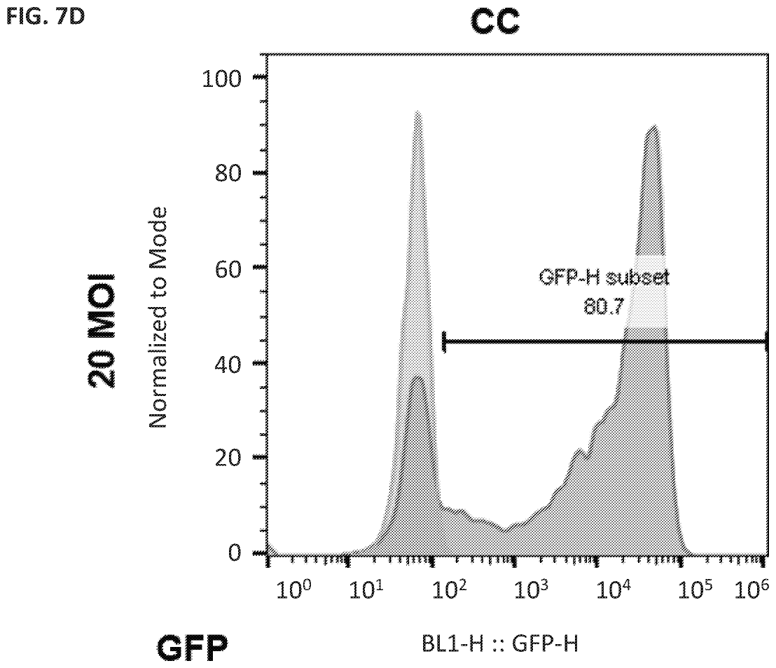

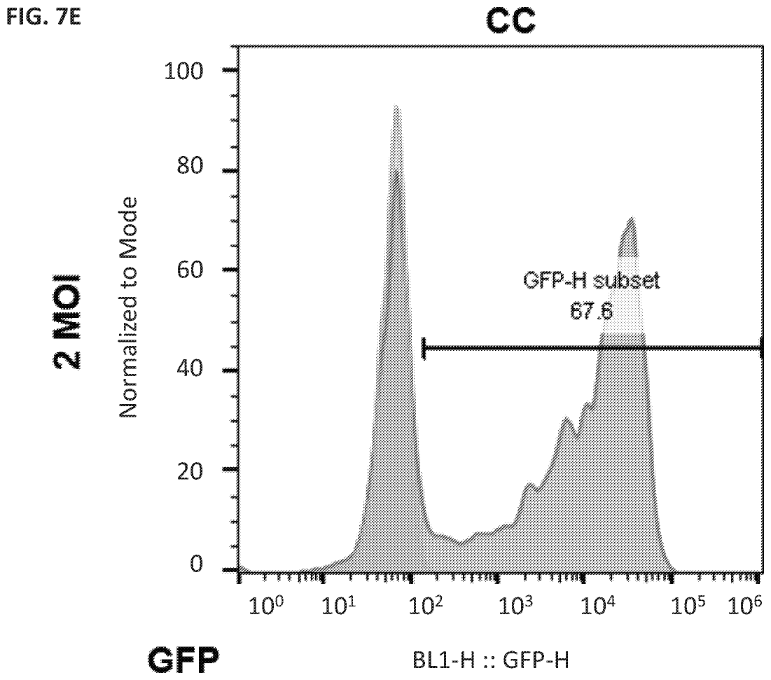

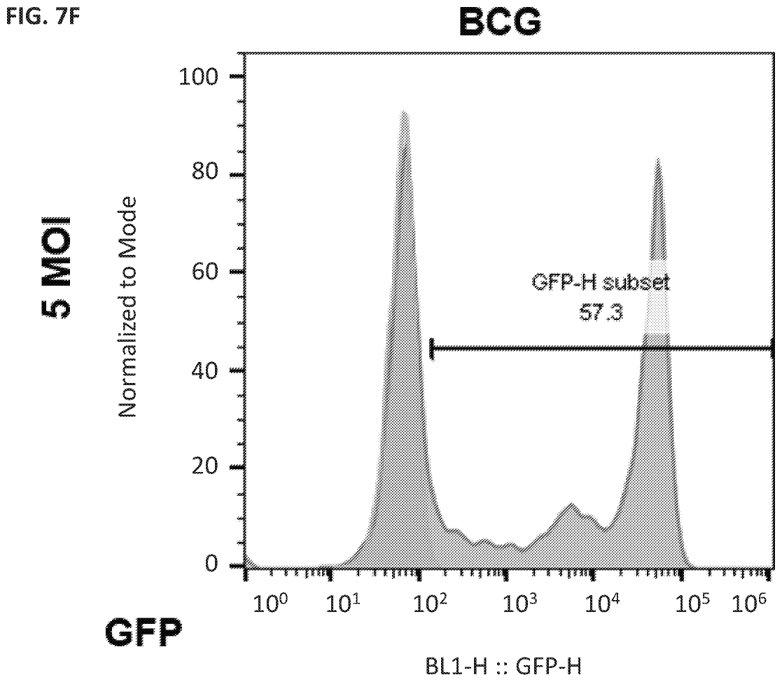

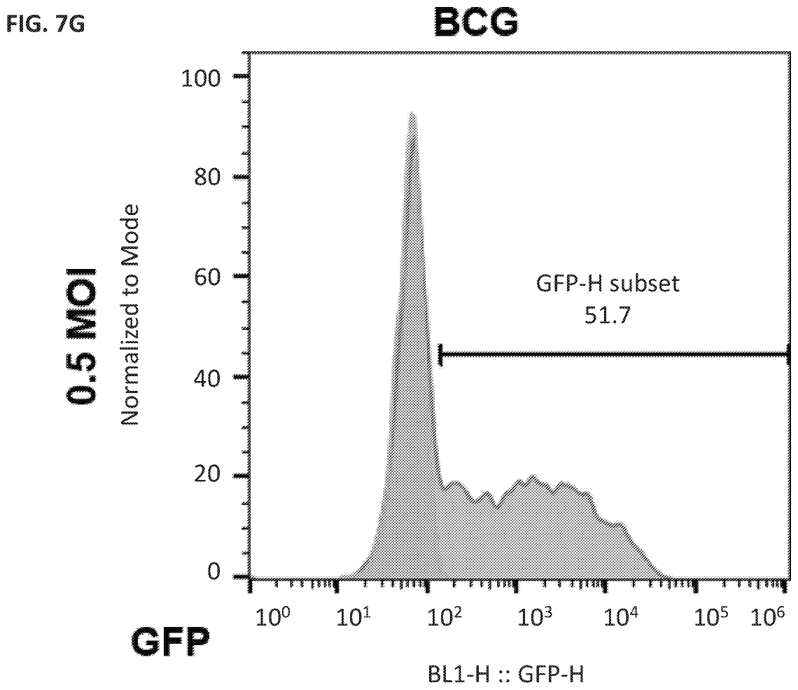

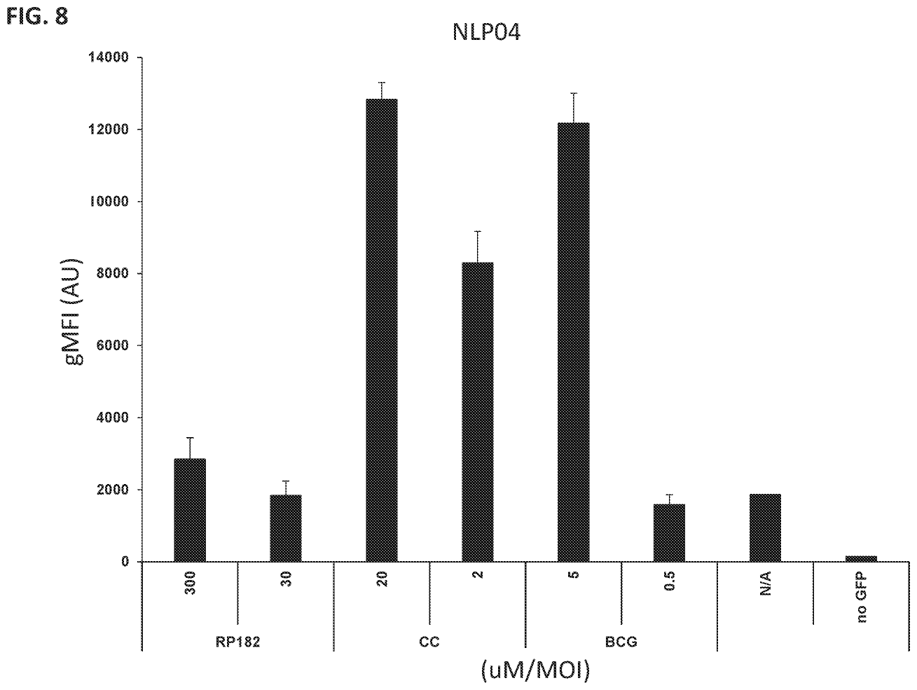

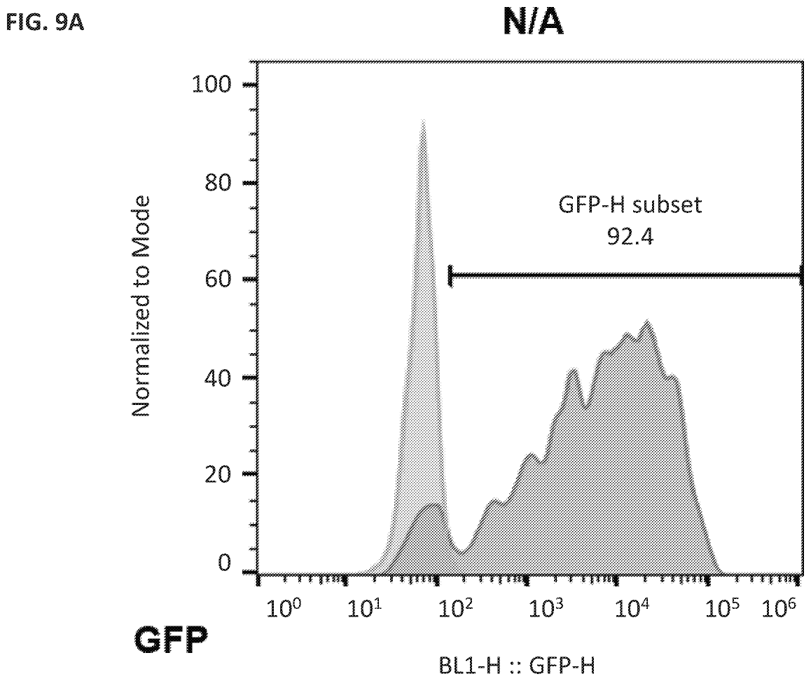

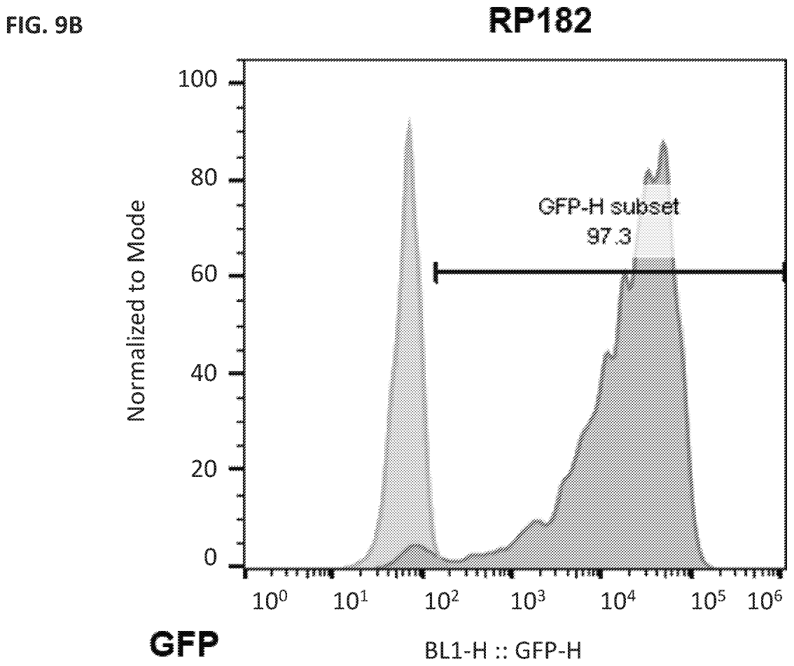

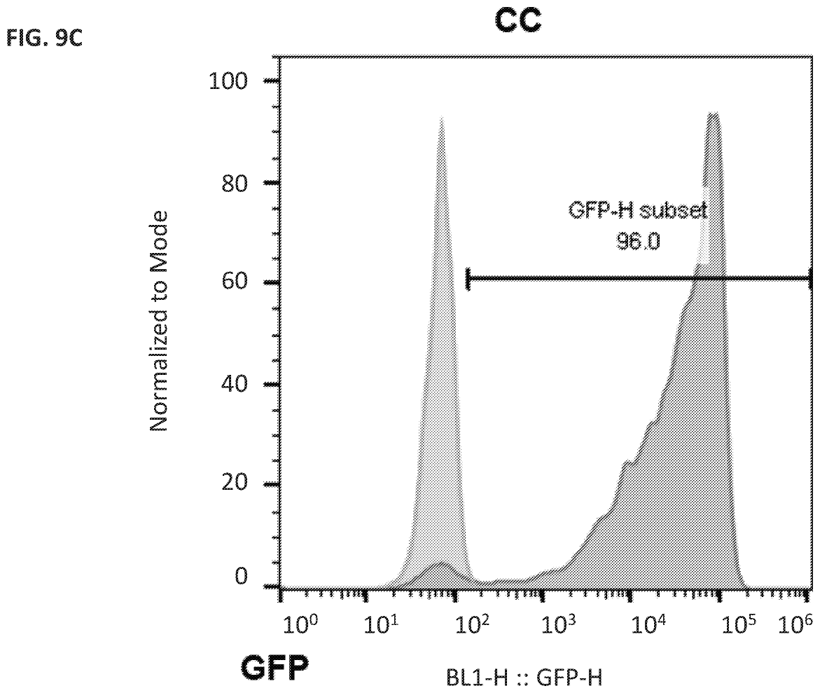

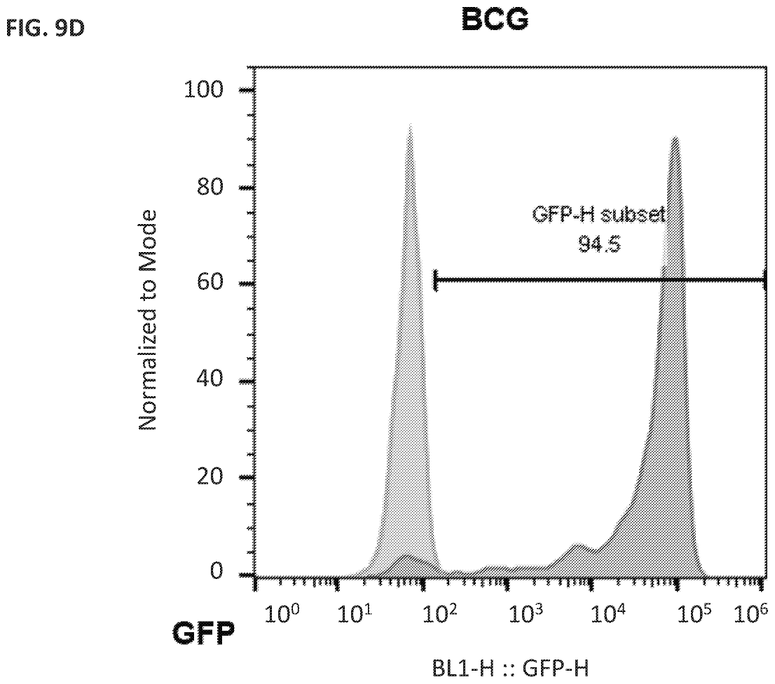

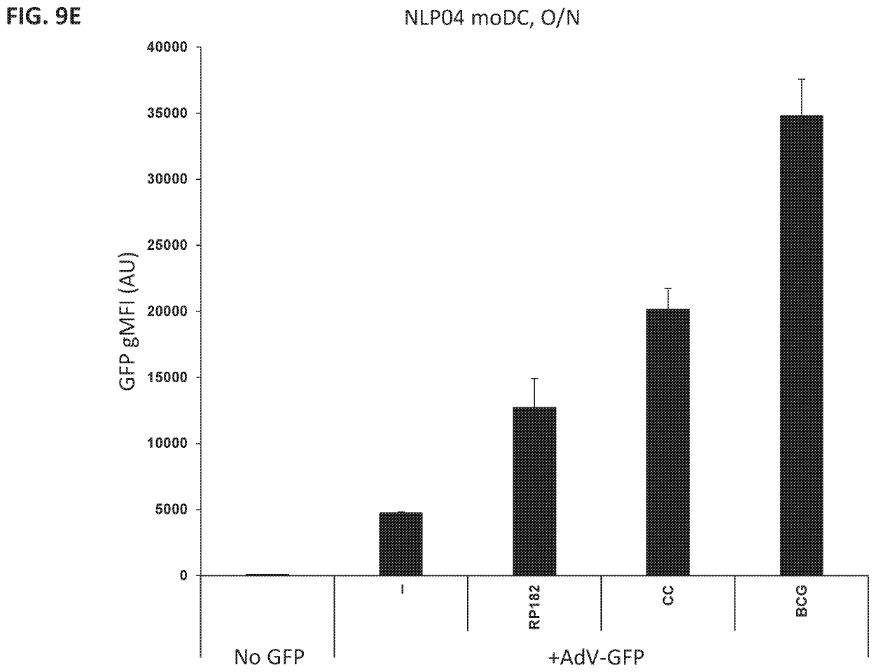

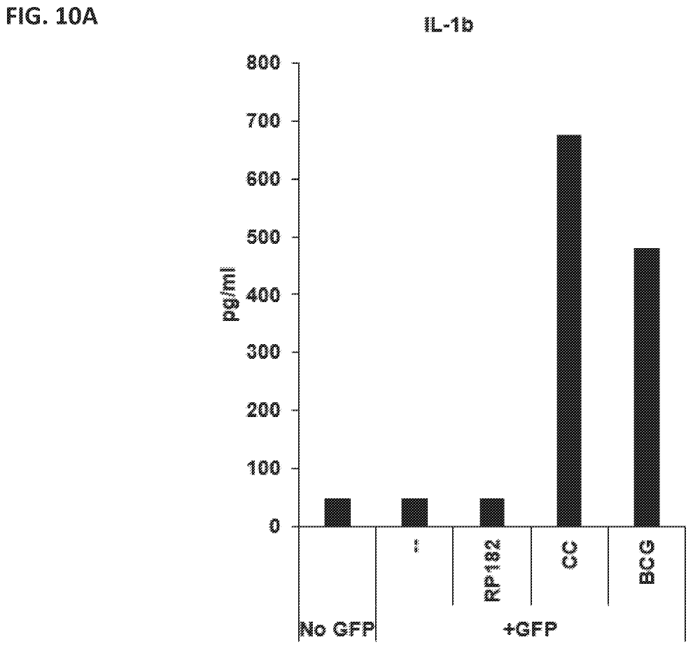

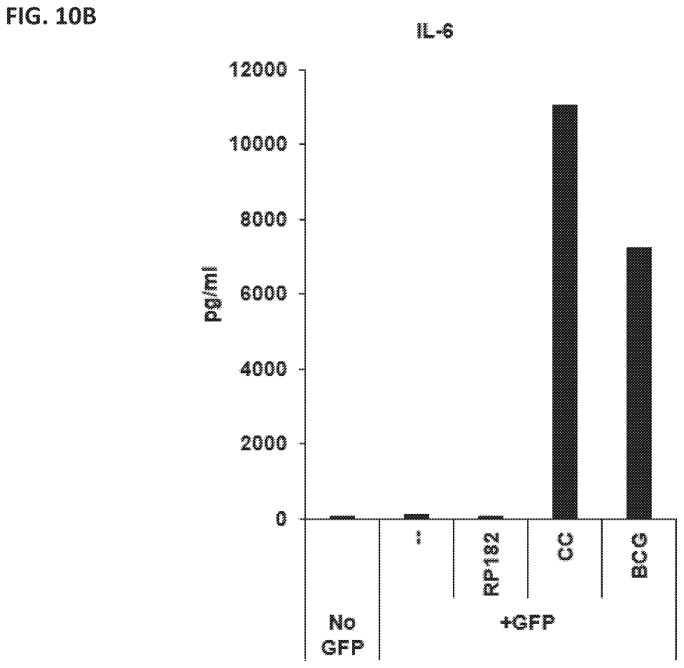

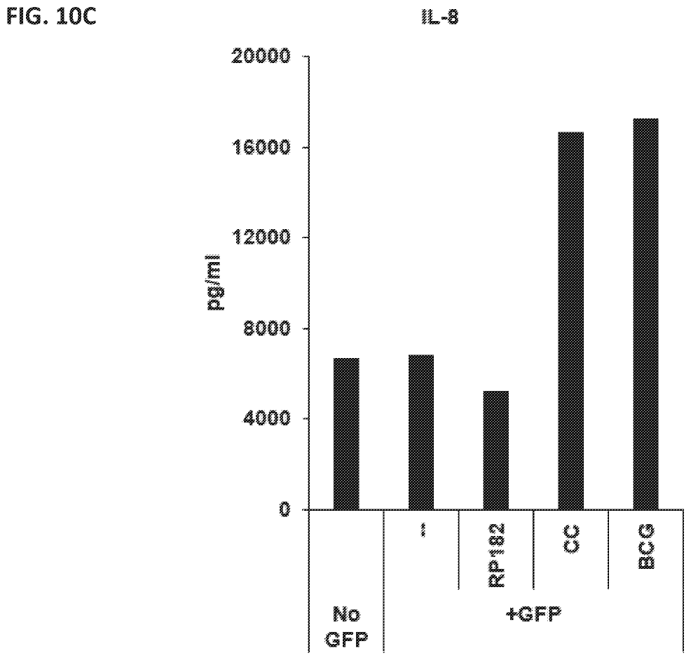

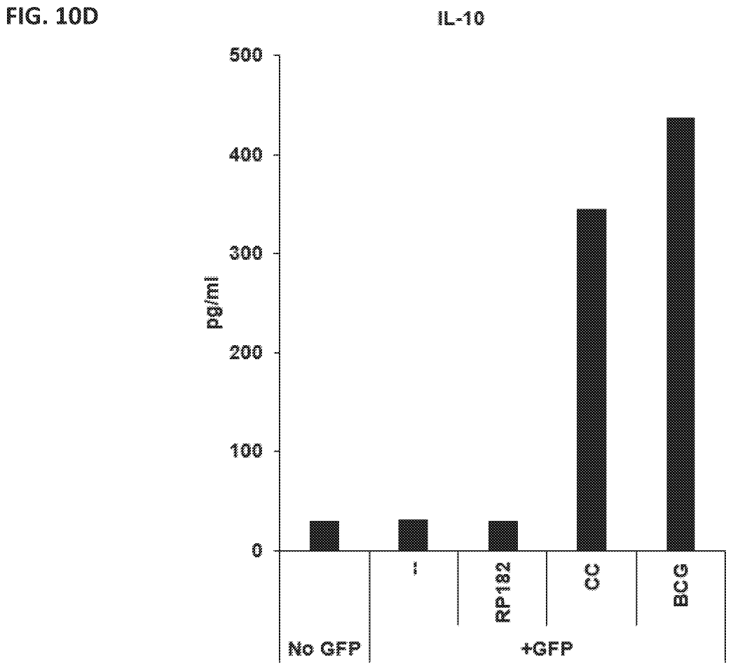

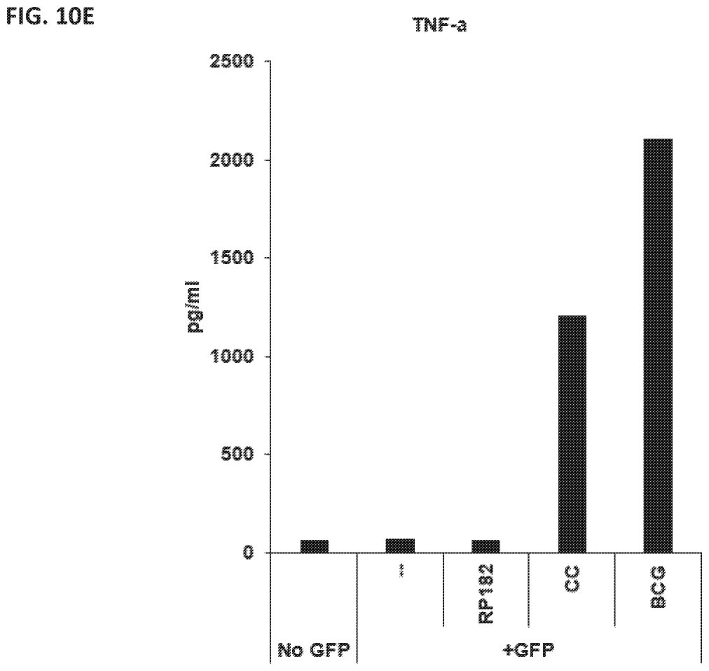

### DETAILED DESCRIPTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

All publications, patents and published patent applications referred to in this application are specifically incorporated by reference herein. In case of conflict, the present specification, including its specific definitions, will control.

The present invention is related to the use of RP182 (also referred to as “RP-182”) and related peptides in vaccines (such as cancer vaccines) to increase antigen uptake and immune activation with fewer potentially dangerous inflammatory side effects. When antigen presenting cells (APCs) are pre-treated with a peptide, such as RP182, prior to exposure to adenovirus encoding green fluorescent protein (GFP), the APCs show much more GFP compared to control cells pre-treated with irrelevant control peptide. The present invention discloses the surprising finding that RP-182 peptide and related peptides increase phagocytosis without enhancing inflammation.

As demonstrated in the figures, each of the RP peptides, the E. coli, and the BCG induce phagocytosis (as indicated in GFP expression well above baseline), but that the CC and BCG also induce inflammation (as shown by the increase in inflammation markers above baseline), while the RP peptides do not. In other words, only the RP peptides induce phagocytosis without inflammation.

The present invention generally relates to pharmaceutical compositions comprising a peptide and an immunotherapeutic composition comprising a viral vector and a nucleic acid sequence encoding an antigen; wherein the peptide is 3 to 24 amino acid residues in length and comprises a striapathic region consisting of alternating hydrophilic and hydrophobic modules; wherein each hydrophilic module consists of from 1 to 5 hydrophilic amino acid residues; and wherein each hydrophobic module consists of from 1 to 5 hydrophobic amino acid residues. The present invention also relates to a method of inducing phagocytosis without inflammation, comprising administering to a subject in need thereof, an immunotherapeutic composition comprising a viral vector and a nucleic acid sequence encoding an antigen; and peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues.

In one aspect, the viral vector is a replication defective adenovirus vector comprising a deletion in an E2b region of the replication defective adenovirus vector and a nucleic acid sequence encoding an antigen. In another aspect, the replication defective adenovirus vector further comprises a deletion in an E1 region of the replication defective adenovirus vector, a deletion in an E3 region of the replication defective adenovirus vector, a deletion in an E4 region of the replication defective adenovirus vector, or a combination thereof.

In still another aspect, the antigen is selected from the group consisting of a cancer associated antigen, and an infectious disease associated antigen.

The present invention also relates to a method of inducing phagocytosis, comprising administering to a subject in need thereof, a composition comprising a yeast lysate prepared from a yeast; and an immunotherapeutic composition comprising viral a vector and a nucleic acid sequence encoding an antigen. In one aspect, the method of inducing phagocytosis does not cause inflammation. In one aspect of the method, the yeast lysate lacks yeast membranes and yeast cell walls. In still another aspect of the method, the yeast lysate comprises intact yeast. In a further aspect, the yeast is heat-inactivated. In yet another aspect, the yeast is selected from the group consisting of Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia hpolytica. In a preferred embodiment, the yeast is Saccharomyces cerevisiae. In yet another aspect, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In another aspect, the antigen is selected from the group consisting of a cancer associated antigen and an infectious disease associated antigen.

The present invention also relates to a method of inducing phagocytosis, comprising administering to a subject in need thereof, a composition comprising a bacterial lysate prepared from a bacteria; and an immunotherapeutic comprising a viral vector and a nucleic acid sequence encoding an antigen. In one aspect, the method of inducing phagocytosis does not cause inflammation. In one aspect, the bacteria is selected from the group consisting of Haemophilus influenzae, Klebsiella pneumoniae, Moraxella catarrhalis, Staphylococcus aureus, Streptococcus mitis, Streptococcus pneumoniae, S. pyogenes, K pneumoniae, M catarrhalis S. pyogenes, S. agalactiae, K. ozaenae, S. viridans, Escherichia coli, S. epidermidis, S. salivarius, Corynebacterium pseudodiphtheriticum. In still another aspect, the bacteria is a gram negative bacteria (such as cyanobacteria, spirochaetes and proteobacteria) In yet another aspect, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In another aspect, the antigen is selected from the group consisting of a cancer associated antigen and an infectious disease associated antigen.

The present invention further relates to a pharmaceutical composition comprising a yeast lysate prepared from a yeast; and a viral vector and a nucleic acid sequence encoding an antigen. In one aspect of the composition, the yeast lysate lacks yeast membranes and yeast cell walls. In still another aspect of the composition, the yeast lysate comprises intact yeast. In a further aspect, the yeast is heat-inactivated. In yet another aspect, the yeast is selected from the group consisting of Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia hpolytica. In a preferred embodiment, the yeast is Saccharomyces cerevisiae. In still another aspect, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In one aspect of the composition, the antigen is selected from the group consisting of a cancer associated antigen and an infectious disease associated antigen.

The present invention further relates to a pharmaceutical composition comprising a bacterial lysate prepared from a bacteria; and a viral vector and a nucleic acid sequence encoding an antigen. In one aspect, the bacteria is selected from the group consisting of Haemophilus influenzae, Klebsiella pneumoniae, Moraxella catarrhalis, Staphylococcus aureus, Streptococcus mitis, Streptococcus pneumoniae, S. pyogenes, K pneumoniae, M catarrhalis S. pyogenes, S. agalactiae, K. ozaenae, S. viridans, Escherichia coli, S. epidermidis, S. salivarius, Corynebacterium pseudodiphtheriticum. In another aspect, the bacteria is a gram-negative bacteria (such as cyanobacteria, spirochaetes and proteobacteria). In still another aspect, the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and wherein each hydrophobic module consists of from 1 to 5 hydrophobic amino acid residues. In one aspect of the composition, the antigen is selected from the group consisting of a cancer associated antigen and an infectious disease associated antigen.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient” and “individual” are used interchangeably and refer to either a human or a non-human animal. These terms include mammals such as humans, primates, livestock animals (e.g., bovines, porcines), companion animals (e.g., canines, felines) and rodents (e.g., mice and rats).

The term “pharmaceutically acceptable carrier” refers to a non-toxic carrier that may be administered to a patient, together with compositions of this invention, and which does not destroy the pharmacological activity of the active agents within the composition. The term “excipient” refers to an additive in a formulation or composition that is not a pharmaceutically active ingredient.

The term “pharmaceutically effective amount” refers to an amount effective to treat a patient, e.g., effecting a beneficial and/or desirable alteration in the general health of a patient suffering from a disease (including but not limited to cancer, viral infection). The skilled worker will recognize that treating includes, but is not limited to, killing cells (such as cancer cells), preventing the growth of new cancer cells, causing tumor regression (a decrease in tumor size), causing a decrease in metastasis, improving vital functions of a patient, improving the well-being of the patient, decreasing pain, improving appetite, improving the patient's weight, and any combination thereof. A “pharmaceutically effective amount” also refers to the amount required to improve the clinical symptoms of a patient.

The invention provides anti-inflammatory polypeptides, which can be referred to as “RP peptides”. The invention also provides anti-inflammatory polypeptides that share a minimum degree of homolog with any of the exemplary RIP peptides disclosed herein and described in US2016/0101150.

The terms “peptide” and “polypeptide” are used synonymously herein to refer to polymers constructed from amino acid residues. The term “amino acid residue ” as used herein refers to any naturally occurring amino acid (L or D form), non-naturally occurring amino acid, or amino acid mimetic (such as peptiod monomer).

The “length” of a polypeptide is the number of amino acid residues linked end-to-end that constitute the polypeptide, excluding any non-peptide linkers and/or modifications that the polypeptide may contain.

The term “striapathic region,” as used herein, refers to an alternating sequence of hydrophobic and hydrophilic modules. A “hydrophobic module” is made up of a peptide sequence consisting of one to five hydrophobic amino acid residues. Likewise, a hydrophilic module is made up of a peptide sequence consisting of one to five hydrophilic amino acid residues.

Hydrophobic amino acid residues are characterized by a functional group (“side chain”) that has predominantly non-polar chemical properties. Such hydrophobic amino acid residues can be naturally occurring (L or D form) or non-naturally occurring. Alternatively, hydrophobic amino acid residues can be amino acid mimetics characterized by a functional group (“side chain”) that has predominantly non-polar chemical properties. Conversely, hydrophilic amino acid residues are characterized by a functional group (“side chain”) that has predominantly polar (charged or uncharged) chemical properties. Such hydrophilic amino acid residues can be naturally occurring (L or D form) or non-naturally occurring. Alternatively, hydrophilic amino acid residues can be amino acid mimetics characterized by a functional group (“side chain”) that has predominantly polar (charged or uncharged) chemical properties. Examples of hydrophilic and hydrophobic amino acid residues are shown in Table 1, below. Suitable non-naturally occurring amino acid residues and amino acid mimetics are known in the art. See, e.g., Liang et al. (2013), “An Index for Characterization of Natural and Non-Natural Amino Acids for Peptidomimetics,” PLoS ONE 8(7):e67844.

Although most amino acid residues can be considered as either hydrophobic or hydrophilic, a few, depending on their context, can behave as either hydrophobic or hydrophilic. For example, due to their relatively weak non-polar characteristics, glycine, proline, and/or cysteine can sometimes function as hydrophilic amino acid residues. Conversely, due to their bulky, slightly hydrophobic side chains, histidine and arginine can sometimes function as hydrophobic amino acid residues.

The term “anti-inflammatory property,” as used herein, refers to any property of a polypeptide that can be evaluated in silico, in vitro, and/or in vivo, that reduces or inhibits, or would be expected to reduce or inhibit, a pro-inflammatory signal mediated by a protein target and/or reduces or inhibits inflammation in a subject.

Peptides of the invention have the following characteristics: a length of 3 to 24 amino acid residues; a striapathic region that comprises at least 25% of the length of the polypeptide; and at least one anti-inflammatory property.

The anti-inflammatory peptide and/or its striapathic region can have a length that is greater than 3 amino acid residues and/or less than 24 amino acid residues. Thus, the requisite length of the polypeptide can be, for example, 3 to 20, 3 to 18, 3 to 16, 3 to 14, 3 to 12, 4 to 20, 4 to 18, 4 to 16, 4 to 14, 4 to 12, 5 to 20, 5 to 18, 5 to 16, 5 to 14, 5 to 12, 6 to 20, 6 to 18, 6 to 16, 6 to 14, 6 to 12, 7 to 20, 7 to 18, 7 to 16, 7 to 14, or in certain embodiments 7 to 12 amino acid residues. For an anti-inflammatory polypeptide that is longer than 12 amino acid residues, it can be advantageous to design a kink in the secondary structure (e.g., such as produced by a proline residue) such that the polypeptide has a striapathic region that is 12 or fewer amino acid residues in length. The striapathic region of an anti-inflammatory peptide can comprise at least 30%, 35%, 40%, 45%, 50%, 60%, 65%, 70%, 75%, 80%, 85%, 90%, 95%, or 100% of the length of the polypeptide.

An anti-inflammatory polypeptide can have a striapathic region that includes at least two hydrophobic modules and one or more (e.g., two or three) hydrophilic modules. Alternatively, an anti-inflammatory polypeptide can have a striapathic region that includes at least three hydrophobic modules and two or more (e.g., three or four) hydrophilic modules; a striapathic region that includes at least two hydrophilic modules and one or more (e.g., two or three) hydrophilic modules; or a striapathic region that includes at least three hydrophilic modules and two or more (e.g., three or four) hydrophobic modules.

Unless otherwise specified, it is to be understood that each embodiment of the invention may be used alone or in combination with any one or more other embodiments of the invention.

The compositions provided herein are useful for a variety of clinical applications. The compositions of the invention may be administered for the treatment of various diseases or conditions or used in the manufacture of a medicament for the treatment of various diseases or conditions. As used herein, the terms “treat,” “treating,” and similar words shall mean stabilizing and/or reducing the symptoms of a disease or condition. In some aspects, the compositions of the invention can be used to prevent the occurrence of a disease or condition or curing a medical condition or disease.

The term “adenovirus” or “Ad” refers to a group of non-enveloped DNA viruses from the family Adenoviridae. in addition to human hosts, these viruses can be found in, but are not limited to, avian, bovine, porcine and canine species. The present invention contemplates the use of any adenovirus from any of the four genera of the family Adenoviridae (e.g., Aviadenovirus, Mastadenovirus, Atadenovirus and Siadeliovirus) as the basis of an E2b deleted virus vector, or vector containing other deletions as described herein. In addition, several serotypes are found in each species. Ad also pertains to genetic derivatives of any of these viral serotypes, including but not limited to, genetic mutation, deletion or transposition of homologous or heterologous DNA sequences.

The term “Adenovirus5 null (Ad5null)”, as used herein, refers to a non-replicating Ad that does not contain any heterologous nucleic acid sequences for expression.

The term “First Generation adenovirus”, as used herein, refers to an 5Ad that has the early region 1 (E1) deleted. In additional cases, the nonessential early region 3 (E3) may also be deleted.

The term “gutted” or “gutless”, as used herein, refers to an adenovirus vector that has been deleted of all viral coding regions.

The term “transfection” as used herein refers to the introduction of foreign nucleic acid into eukaryotic cells. Transfection may be accomplished by a variety of means known to the art including calcium phosphate-DNA co-precipitation, DEAE-dextran-mediated transfection, polybrene-mediated transfection, electroporation, microiniection, liposome fusion, lipofection, protoplast fusion, retroviral infection, and biolistics.

The term “stable transfection” or “stably transfected” refers to the introduction and integration of foreign nucleic acid, DNA or RNA, into the genome of the transfected cell. The term “stable transfectant” refers to a cell Which has stably integrated foreign DNA into the genomic DNA.

The term “Second Generation Adenovirus”, as used herein, refers to an Ad that has all or parts of the E1, E2, E3, and, in certain embodiments, E4 DNA gene sequences deleted (removed) from the virus. Compared to First Generation adenovirus vectors, certain embodiments of the Second Generation E2b deleted adenovirus vectors of the present invention contain additional deletions in the DNA polymerase gene (pol) and deletions of the pre-terminal protein (pTP).

Thus, the present invention contemplates the use of E2b deleted adenovirus vectors, such as those described in U.S. Pat. Nos. 6,063,622; 6,451,596; 6,057,158; and 6,083,750.

Antigens of the present invention include but are not limited to antigens derived from any of a variety of infectious agents or cancer cells. As used herein, an “infectious agent” is any living organism capable of infecting a host and “cancer” means a neoplastic cell. Infectious agents include, for example, bacteria, any variety of viruses, such as, single stranded RNA viruses, single stranded DNA viruses, fungi, parasites, and protozoa. Examples of infectious agents include, but are not limited to, Actinobacillus spp., Actinomyces spp., Adenovirus (types 1, 2, 3, 4, 5 et 7), Adenovirus (types 40 and 41), Aerococcus spp., Aeromonas hydrophila, Ancylostoma duodenale, Angiostrongylus cantonensis, Ascaris lumbricoides, Ascaris spp., Aspergillus spp., Babesia spp, B. microti, Bacillus anthracis, Bacillus cereus, Bacteroides spp., Balantidium coli, Bartonella bacilliformis, Blastomyces dermatitidis, Bluetongue virus, Bordetella bronchiseptica, Bordetella pertussis, Borrelia afzelii, Borrelia burgdorferi, Borrelia garinii, Branhamella catarrhalis, Brucella spp. (B. abortus, B. canis, B. melitensis, suis), Brugia spp., Burkholderia, (Pseudomonas) mallei, Burkholderia (Pseudomonas) pseudomallei, California serogroup, Campylobacter fetus subsp. Fetus, Campylobacter jejuni, C. coli, C. fetus subsp. Jejuni, Candida albicans, Capnocytophaga spp., Chi kungunya virus, Chlamydia psittaci, Chlamydia trachomatis, Citrobacter spp., Clonorchis sinensis, Clostridium botulinum, Clostridium difficile, Clostridium perfringens, Clostridium tetani, Clostridium spp. (with the exception of those species listed above), Coccidioides brunitis, Colorado tick fever virus, Corynebacterium diphtheriae, Coxiella burnetii, Coxsackievirus, Creutzfeldt-Jakob agent, Kuru agent, Crimean-Congo hemorrhagic fever virus, Cryptococcus neoformans, Cryptosporidium parvum, Cytomegalovirus, Cyclospora cayatanesis, Dengue virus (1, 2, 3, 4), Diphtheroids, Eastern (Western) equine encephalitis virus, Ebola virus, Echinococcus granulosus, Echinococcus multilocularis, Echovirus, Edwardsiella tarda, Entamoeba histolytica, Enterobacter spp., Enterovirus 70, Epidermophyton floccosum, Ehrlichia spp, Ehrlichia sennetsu, Microsporum spp., Trichophyton spp., Epstein-Barr virus, Escherichia coli, enterohemorrhagic, Escherichia coli, enteroinvasive, Escherichia coli, enteropathogenic, Escherichia coli, enterotoxigenic, Fasciola hepatica, Francisella tularensis, Fusobacterium spp., Gemella haemolysans, Giardia lamblia, Guanarito virus, Haemophilus ducreyi, Haemophilus influenzae (group b), Hantavirus, Hepatitis A virus, Hepatitis B virus, Hepatitis C virus, Hepatitis D virus, Hepatitis E virus, Herpes simplex virus, Herpesvirus simiae, Histoplasma capsulatum, Human coronavirus, Human immunodeficiency virus, Human papillomavirus, Human rotavirus, Human T-lymphotrophic virus, influenza virus including H5N1, Junin virus/Machupo virus, Kiebsiella spp., Kyasanur Forest disease virus, Lactobacillus spp., Lassa virus, Legionella pneumophila, Leishmania major, Leishmania infantum, Leishmania spp., Leptospira interrogans, Listeria monocytogenes, Lymphocytic choriomeningitis virus, Machupo virus, Marburg virus, Measles virus, Micrococcus spp., Moraxella spp., Mycobacterium spp. (other than M. bovis, M. tuberculosis, M. avium, M. leprae), Mycobacterium tuberculosis, M. Bovis, Mycoplasma hominis, M. orale, M. salivarium, M. fermentans, Mycoplasma pneumoniae, Naegleria fowleri, Necator americanus, Neisseria gonorrhoeae, Neisseria meningitides, Neisseria spp. (other than N. gonorrhoeae and N. meningitidis), Nocardia spp., Norwalk virus, Ornsk hemorrhagic fever virus, Onchocerca volvulus, Opisthorchis spp., Parvovirus B19, Pasteurella spp., Peptococcus spp., Peptostreptococcus spp., Plasmodium falciparum, Plasmodium vivax, Plasmodium spp., Plesiomonas shigelloides, Powassan encephalitis virus, Proteus spp., Pseudomonas spp. (other than P. mallei, P. pseudomallei), Rabies virus, Respiratory syncytial virus, Rhinovirus, Rickettsia akari, Rickettsia prowazekii, R. Canada, Rickettsia rickettsii, Rift Valley virus, Ross river virus/O'Nyong-Nyong virus, Rubella virus, Salmonella choieraesuis, Salmonella paratyphi, Salmonella typhi, Salmonella spp. (with the exception of those species listed above), Schistosoma spp., Scrapie agent, Serratia spp., Shigella spp., Sindbis virus, Sporothrix schenckii, St. Louis encephalitis virus, Murray Valley encephalitis virus, Staphylococcus aureus, Streptobacillus moniliformis, Streptococcus agalactiae, Streptococcus faecalis, Streptococcus pneumoniae, Streptococcus pyogenes, Streptococcus salivarius, Taenia saginata, Taenia solium, Toxocara canis, T. cati, T. cruzi, Toxoplasma gondii, Treponema pallidum, Trichinella spp., Trichomonas vaginalis, Trichuris trichiura, Trypanosoma brucei, Trypanosoma cruzi, Ureaplasina urealyticum, Vaccinia virus, Varicella-zoster eastern equine encephalitis virus (EEEV), severe acute respiratory virus (SARS), Venezuelan equine encephalitis virus (VEEV), Vesicular stomatitis virus, Vibrio cholerae, serovar 01, Vibrio parahaemolyticus, West Nile virus, Wuchereria bancrofti, Yellow fever virus, Yersinia enterocolitica, Yersinia pseudotuberculosis, and Yersinia pestis.

Examples of infectious agents associated with human malignancies include Epstein-Barr virus, Helicobacter pylori, Hepatitis B virus, Hepatitis C virus, Human heresvirus-8, Human immunodeficiency virus, Human papillomavirus, Human T cell leukemia virus, liver flukes, and Schistosoma haematobium.

Antigens may include proteins, or variants or fragments thereof, produced by any of the infectious organisms described herein, such as, but not limited to, viral coat proteins, i.e., influenza neuraminidase and hemagglutinin, HIV gp160 or derivatives thereof, HIV Gag, HIV Nef, HIV Pol, SARS coat proteins, herpes virion proteins, WNV proteins, etc. Target antigens may also include bacterial surface proteins including pneumococcal PsaA, PspA, LytA, surface or virulence associated proteins of bacterial pathogens such as Nisseria gonnorhea, outer membrane proteins or surface proteases.

Antigens may also include proteins, or variants or fragments thereof, of infectious agents associated with human malignancies such as the human papillomavirus (HPV) oncoproteins E6 and E7.

Antigens of the present invention include but are not limited to antigens derived from a variety of tumor proteins. Illustrative tumor proteins useful in the present invention include, but are not limited to any one or more of, WTI, HPV E6, HPV E7, p53, MAGE-A 1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MCIR, Gp100, PSA, PSA, Tyrosinase, TRP-1, TRP-2, ART-4, CAMEL, CEA, Cyp-B, Her2/neu, BRCA1, hTERT, hTRT, iCE, MUC1, MUC2, PRAME, P15, RU1, RU2, SART-1, SART-3, WT1, AFP, p-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TP1/mbcr-abl, ETV6/AML, LDLR/FUT, Pml/RARa, and TEL/AML 1. These and other tumor proteins are known to the skilled artisan.

Methods are also provided for treating or ameliorating the symptoms of any of the infectious diseases or cancers as described herein. The methods of treatment comprise administering the compositions of the invention one or more times to individuals suffering from or at risk from suffering from an infectious disease or cancer as described herein. As such, the present invention provides methods for vaccinating against infectious diseases or cancers in individuals Who are at risk of developing such a disease. individuals at risk may be individuals who may be exposed to an infectious agent at some time or have been previously exposed but do not yet have symptoms of infection or individuals having a genetic predisposition to developing a cancer or being particularly susceptible to an infectious agent.

Routes and frequency of administration of the therapeutic compositions described herein, as well as dosage, will vary from individual to individual, and from disease to disease, and may be readily established using standard techniques. In general, the pharmaceutical compositions and vaccines may be administered by injection (e.g., intracutaneous, intramuscular, intravenous or subcutaneous), intranasally (e.g., by aspiration), in pill form (e.g. swallowing, suppository for vaginal or rectal delivery).

As described herein, the compositions of the invention are suitable for parenteral administration. These compositions may be administered, for example, intraperitoneally, intravenously, or intrathecally, parenterally, orthotopi rally, subcutaneously, topically, nasally, orally, sublingually, intraocularly, by means of an implantable depot, using nanoparticle-based delivery systems, microneedle patch, microspheres, beads, osmotic or mechanical pumps, and/or other mechanical means. One of skill in the art would appreciate that a method of administering the composition of the invention would depend on factors such as the age, weight, and physical condition of the patient being treated, and the disease or condition being treated. The skilled worker would, thus, be able to select a method of administration optimal for a patient on a case-by-case basis.

In conjunction with any of the foregoing methods, the compositions can be administered in combination with another drug. In each case, the composition of the invention can be administered prior to, at the same time as, or after the administration of the other drug. For the treatment of cancer, the injectable compositions of the invention can be administered in combination with a chemotherapeutic agent selected from the group consisting of steroids, anthracyclines, thyroid hormone replacement drugs, thymidylate-targeted drugs, Chimeric Antigen Receptor/T cell therapies, and other cell therapies. Specific chemotherapeutic agents include, for example, Gemcitabine, Docetaxel, Bleomycin, Erlotinib, Gefitinib, Lapatinib Imatinib, Dasatinib Nilotinib Bosutinib, Crizotinib, Ceritinib, Trametinib, Bevacizumah, Sunitinib, Sorafenib, Trastuzurnab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouraci1, Teysumo, Paclitaxel, Prednisone, Levothyroxine, and Pemetrexed.

Alternatively, for the methods of treating cancer, the compositions of the invention can be administered in combination with radiation therapy or can be administered prior to, or after the administration of the radiation therapy.

In some embodiments, it may be beneficial to include one or more excipients in a composition of the invention. One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to empirically determine which excipients, if any, to include in the formulations or compositions of the invention. Excipients of the invention may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some embodiments, it may be beneficial to include a pharmaceutically acceptable carrier in the compositions of the invention.

In some embodiments, it may be beneficial to include a solubilizing agent in the compositions of the invention. Solubilizing agents may be useful for increasing the solubility of any of the components of the formulation. or composition, including RP-182 or an excipient. The solubilizing agents described herein are not intended to constitute an exhaustive list, but are provided merely as exemplary solubilizing agents that may be used in the formulations or compositions of the invention. In certain embodiments, solubilizing agents include, but are not limited to, ethyl alcohol, tert butyl alcohol, polyethylene glycol, glycerol, methylparaben, propylparaben, polyethylene glycol, polyvinyl pyrrolidone, and any pharmaceutically acceptable salts and/or combinations thereof.

The pH of the compositions of the invention may be any pH that provides desirable properties for the composition. Desirable properties may include, for example, peptide (such as RP-182) stability, increased peptide retention as compared to compositions at other pHs, and improved filtration efficiency.

In some embodiments, it may be beneficial to include a tonicity-adjusting agent in the compositions a the invention. The tonicity of a liquid composition is an important consideration when administering the composition to a patient, for example, by parenteral administration. Tonicity-adjusting agents, thus, may be used to help make a composition suitable for administration. Tonicity-adjusting agents are well known in the art. Accordingly, the tonicity-adjusting agents described herein are not intended to constitute an exhaustive list but are provided merely as exemplary tonicity-adjusting agents that may be used in the formulations or compositions of the invention. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

In some embodiments, it may be beneficial to include a stabilizing agent in the compositions of the invention. Stabilizing agents help increase the stability of peptides (such as RP-182) in compositions of the invention.

In some embodiments, it may be beneficial to include a protectant in the compositions of the invention. Protectants are agents that protect a pharmaceutically active ingredient (e.g., RP-182) from an undesirable condition (e.g., instability caused by freezing lyophilization or oxidation). Protectants can include, for example, cryoprotectants, lyoprotectants, and antioxidants. Cryoprotectants are useful in preventing loss of potency of an active pharmaceutical ingredient (e.g., RP-182) when a formulation is exposed to a temperature below its freezing point. For example, a cryoprotectant could be included in a reconstituted lyophilized formulation of the invention so that the formulation could be frozen before dilution for intravenous (IV) administration. Cryoprotectants are well known in the art. Accordingly, the cryoprotectants described herein are not intended to constitute an exhaustive list, but are provided merely as exemplary cryoprotectants that may be used in the formulations or compositions of the invention. Cryoprotectants include, but are not limited to, solvents, surfactants, encapsulating agents, stabilizing agents, viscosity modifiers, and combinations thereof. Cryoprotectants may include, for example, disaccharides (e.g., sucrose, lactose, maltose, and trehalose), polyols (e.g., glycerol, mannitol, sorbitol, and dulcitol), glycols (e.g., ethylene glycol, polyethylene glycol, propylene.

Lyoprotectants are useful in stabilizing the components of a lyophilized formulation or composition. For example, a peptide such as RP 182 could be lyophilized with a. lyoprotectant prior to reconstitution. Lyoprotectants are well known in the art. Accordingly, the lyoprotectants described herein are not intended to constitute an exhaustive list, but are provided merely as exemplary lyoprotectants that may be used in the formulations or compositions of the invention. Lyoprotectacts include, but are not limited to, solvents, surfadants, encapsulating agents, stabilizing agents, viscosity modifiers, and conibinatiorrs thereof. Exemplary lyoprotectants may be, for example, sugars and polyols, Trehalose, sucrose, dextran, and hydroxypropyl-beta-cyclodextrin are non-limiting examples of lyoprotectants.

Antioxidants are useful in preventing oxidation of the components of a composition. Oxidation may result in aggregation of a drug product or other detrimental effects to the purity of the drug product or its potency. Antioxidants are well known in the art.

Accordingly, the antioxidants described herein are not intended to constitute an exhaustive list but are provided merely as exemplary antioxidants that may be used in the formulations or compositions of the invention. Antioxidants may be, for example, sodium ascorbate, citrate, thiols, metabisulfite, and combinations thereof.

Variations, modifications, and other implementations of what is described herein will occur to those of ordinary skill without departing from the spirit and the scope of the invention. Accordingly, the invention is not to be limited only to the preceding illustrative description.

Each of the embodiments of the invention may be combined individually or in combination with one or more other embodiments of the invention.

Those skilled in the art will recognize or be able to ascertain using no more than routine experimentation, numerous equivalents to the compounds, compositions, and methods of use thereof described herein. Such equivalents are considered to be within the scope of the invention.

The contents of all references, patents and published patent applications cited throughout this Application, as well as their associated figures are hereby incorporated by reference in their entirety.

1. A pharmaceutical composition comprising a peptide and an immunotherapeutic composition comprising a viral vector and a nucleic acid sequence encoding an antigen;
wherein the peptide is 3 to 24 amino acid residues in length and comprises a striapathic region consisting of alternating hydrophilic and hydrophobic modules;
wherein each hydrophilic module consists of from 1 to 5 hydrophilic amino acid residues; and
wherein each hydrophobic module consists of from 1 to 5 hydrophobic amino acid residues.

2. The pharmaceutical composition of claim 1, wherein the viral vector is a replication defective adenovirus vector comprising a deletion in an E2b region of the replication defective adenovirus vector and a nucleic acid sequence encoding an antigen.

3. The pharmaceutical composition of claim 1, wherein the antigen is selected from the group consisting of a cancer associated antigen and an infectious disease associated antigen.

4. The pharmaceutical composition of claim 2, wherein the replication defective adenovirus vector further comprises a deletion in an E1 region of the replication defective adenovirus vector, a deletion in an E3 region of the replication defective adenovirus vector, a deletion in an E4 region of the replication defective adenovirus vector, or a combination thereof.

5. A method of inducing phagocytosis without inflammation, comprising administering to a subject in need thereof,
a) an immunotherapeutic composition comprising a viral vector and a nucleic acid sequence encoding an antigen; and
b) peptide, wherein the peptide is 3 to 24 amino acid residues in length and comprises a striapathic region consisting of alternating hydrophilic and hydrophobic modules,
wherein each hydrophilic module consists of from 1 to 5 hydrophilic amino acid residues; and
wherein each hydrophobic module consists of from 1 to 5 hydrophobic amino acid residues.

6. The method of claim 5, wherein the viral vector is a replication defective adenovirus vector comprising a deletion in an E2b region of the replication defective adenovirus vector and a nucleic acid sequence encoding an antigen.

7. The method of claim 6, wherein the antigen is selected from the group consisting of a cancer associated antigen and an infectious disease associated antigen.

8. The method of claim 6, wherein the replication defective adenovirus vector further comprises a deletion in an E1 region of the replication defective adenovirus vector, a deletion in an E3 region of the replication defective adenovirus vector, a deletion in an E4 region of the replication defective adenovirus vector, or a combination thereof.

9. A method of inducing phagocytosis, comprising administering to a subject in need thereof,
a) a composition comprising a yeast lysate prepared from a yeast; and
b) an immunotherapeutic composition comprising a viral vector and a nucleic acid sequence encoding an antigen.

10. The method of claim 9, wherein the method of inducing phagocytosis does not cause inflammation.

11. The method of claim 9, wherein the yeast lysate lacks yeast membranes and yeast cell walls.

12. The method of claim 9, wherein the yeast lysate comprises intact yeast.

13. The method of claim 9, wherein the yeast is heat-inactivated.

14. The method of claim 9 any of claims 9 13, wherein the yeast is selected from the group consisting of Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica.

15. The method of claim 14, wherein the yeast is Saccharomyces cerevisiae.

16-19. (canceled)

20. The method of claim 9, wherein the compositing further comprises a peptide, wherein the peptide is 3 to 24 amino acid residues in length and comprises a striapathic region consisting of alternating hydrophilic and hydrophobic modules, wherein each hydrophilic module consists of from 1 to 5 hydrophilic amino acid residues; and
wherein each hydrophobic module consists of from 1 to 5 hydrophobic amino acid residues.

21. The method of claim 9, wherein the antigen is selected from the group consisting of a cancer associated antigen and an infectious disease associated antigen.

22-32. (canceled)
